(19) United States
(12) Patent Application Publication
(10) Pub. No.: US 2016/0082019 A1
(43) Pub. Date: Mar. 24, 2016

(54) Drug Combinations to Treat Cancer

(71) Applicant: EXELIXIS, INC., South San Fancisco, CA (US)

(72) Inventors: Christopher J. Sweeney, Waban, MA (US); Phili W. Kantoff, Brookline, MA (US)

(21) Appl. No.: 14/782,493

(22) PCT Filed: Apr. 4, 2014
(86) PCT No.: PCT/US2014/033008

§ 371 Date: Oct. 5, 2015

Related U.S. Application Data
(60) Provisional application No. 61/808,516, filed on Apr. 4, 2013.

(51) Int. Cl. A61K 31/58; A61K 31/573; A61K 31/47

(52) U.S. Cl. CPC A61K 31/58; A61K 31/47; A61K 31/573

(57) ABSTRACT

This invention relates to the combination of cabozantinib and abiraterone to treat cancer, particularly castration resistant prostate cancer.

### PRIORITY CLAIM

This application claims priority to U.S. Ser. No. 61/808,516, filed Apr. 4, 2013. The entire contents of the aforementioned application are incorporated herein.

### FIELD OF INVENTION

This invention relates to the combination of cabozantinib and abiraterone to treat cancer, particularly castration resistant prostate cancer.

### BACKGROUND OF THE INVENTION

Prostate cancer is made up of an amalgam of clinical states which each have their own unique characteristics. There were approximately 230,000 new diagnoses of prostate cancer in the United States in 2010. Each year, it is estimated that nearly 30,000 men die from castration resistant prostate cancer in the United States alone.

This statistic is mitigated by significant advances that have been made in the therapeutic management of prostate cancer. The advances can be divided into therapies related to hormonal therapy and therapies related to immunotherapy and cytotoxic chemotherapy. With respect to hormonal therapy such as androgen deprivation therapy (ADT) for overt metastatic disease, data shows that combined androgen blockade or so-called CAB (i.e., castration with either orchiectomy or luteinizing-hormone-releasing hormone, hereinafter “LHRH,” analogue plus a non-steroidal anti-androgen) is slightly more effective than castration alone. Data further indicates that castration is probably more effective than anti-androgen therapy alone. While the absolute benefit of ADT over no therapy has not been determined, it is safe to assume there is a substantial benefit. With respect to cytotoxic chemotherapy, the use of docetaxel and more recently cabazitaxel has demonstrated the ability to palliate and increase the chances a man will live longer when treated for castrate resistant prostate cancer (CRPC).

In spite of these advances, there is still room for improvement. There is also still a need for the development of novel systemic therapies for prostate cancer.

### SUMMARY OF THE INVENTION

These and other needs are met by the present invention, which is directed to a method of treating cancer, comprising administering a patient in need of such treatment a compound of formula I:

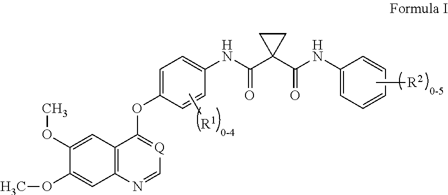

or a pharmaceutically acceptable salt thereof or a pharmaceutical composition comprising the compound of formula I and a pharmaceutically acceptable carrier, wherein:

R¹ is halo;

R² is halo; and

Q is CH or N;

in combination with compound 2:

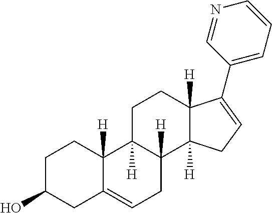

or a pharmaceutically acceptable salt thereof or a pharmaceutical composition comprising compound 2 and a pharmaceutically acceptable carrier.

Another aspect is directed to a method of treating castration resistant prostate cancer, comprising administering a patient in need of such treatment compound 1:

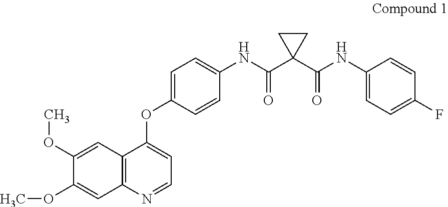 [SMILES: COc1cc2nccc(Oc3ccc(NC(=O)C4(C(=O)Nc5ccc(F)cc5)CC4)cc3)c2cc1OC]

or a pharmaceutically acceptable salt thereof or a pharmaceutical composition comprising compound 1 and a pharmaceutically acceptable carrier;

in combination with a compound 2:

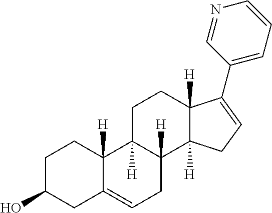

or a pharmaceutically acceptable salt thereof or a pharmaceutical composition comprising compound 2 and a pharmaceutically acceptable carrier.

In another aspect, the invention comprises a pharmaceutical dosage form comprising a compound of formula I or compound 1 with compound 2.

### SUMMARY OF THE FIGURES

FIG. 1 shows the whole body ¹⁸F-FDG PET/CT scans at baseline and 8 weeks following the first dose of study treatment for a 55-year old man with castrate-resistant prostate cancer. This patient was from the 60 mg cabozantinib cohort.

FIG. 2 shows the whole body ¹⁸F—NaF PET/CT scans for the same patient as described in FIG. 1.

FIG. 3 shows the whole body bone scans for the same patient as described in FIG. 1.

FIG. 4 depicts the baseline PET imaging results for ¹⁸F-FDG PET/CT.

FIG. 5 depicts the baseline PET imaging for ¹⁸F—NaF PET/CT.

FIG. 6 shows a preliminary analysis that demonstrated an increase of ¹⁸F—NaF PET/CT SUVmax at 8 weeks. FIG. 6A shows the absolute change in ¹⁸F—NaF PET/CT SUVmax at 8 weeks. FIG. 6B shows the percent change of ¹⁸F—NaF PET/CT SUVmax from baseline at 8 weeks.

FIG. 7 depicts the pharmacokinetic data for cabozantinib in chart of the mean concentration of cabozantinib versus the study day.

FIG. 8 shows ¹⁸F-FDG PET/CT and ¹⁸F—NaF PET/CT scans at baseline and 8 weeks after the first dose for patient 1 of the first cohort, a 75-year old man who received 20 mg cabozantinib.

FIG. 9 shows full body bone scans at baseline, 8 months after the first dose, and 16 weeks after the first dose for the same patient as described in FIG. 8.

FIG. 10 shows ¹⁸F-FDG PET/CT and ¹⁸F—NaF PET/CT scans at baseline and 8 weeks after the first dose for patient 3 of the first cohort, a 52-year old man who received 20 mg cabozantinib.

FIG. 11 shows full body bone scans at baseline and 8 months after the first dose. For the same patient as described in FIG. 10.

### DETAILED DESCRIPTION

As indicated above, the invention is directed to a method of treating cancer, comprising administering a compound of formula I or compound 1 in combination with compound 2.

Compound 1 is known by its chemical name N-(4-{[6,7-bis(methyloxy)quinolin-4-yl]oxy}phenyl)-N′-(4-fluorophenyl)cyclopropane-1,1-dicarboxamide and by the name cabozantinib (COMETRIQ™). Cabozantinib is formulated as the L-malate salt of N-(4-{[6,7-bis(methyloxy)quinolin-4-yl]oxy}phenyl)-N′-(4-fluorophenyl)cyclopropane-1,1-dicarboxamide. WO 2005/030140, the entire contents of which is incorporated herein by reference, discloses compound 1 and describes how it is made and also discloses the therapeutic activity of this compound to inhibit, regulate, and/or modulate the signal transduction of kinases (Assays, Table 4, entry 289). In November 2012, cabozantinib achieved regulatory approval in the United States for the treatment of progressive metastatic medullary thyroid cancer. WO 2005/030140 describes the synthesis of cabozantinib (Example 48) and also discloses the therapeutic activity of this molecule to inhibit, regulate, and/or modulate the signal transduction of kinases (Assays, Table 4, entry 289). Example 48 begins at paragraph [0353] in WO 2005/030140. Information for Compound 1 is available from the FDA at acessdata.fda.gov/scripts/cder/drugsatfda/index.cfin?fuseaction=Search.DrugDetails (last visited Mar. 31, 2013).

Compound 2 is known by the name (3β)-17-(pyridin-3-yl)androsta-5,16-dien-3-ol and by the name abiraterone (Zytiga®). Compound 2 achieved regulatory approval in the United States for the treatment of castration resistant prostate cancer. It is formulated as the prodrug abiraterone acetate.

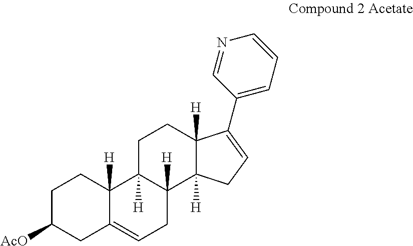

Information for Compound 2 is available from the FDA at accessdata.fda.gov/scripts/cder/drugsatfda/index.cfm?fuseaction=Search.DrugDetails (last visited Mar. 31, 2013).

In these and other embodiments, the compound of formula I or compound 1, or a pharmaceutically acceptable salt thereof, is administered as a pharmaceutical composition, wherein the pharmaceutical composition additionally comprises a pharmaceutically acceptable carrier, excipient, or diluent. In a specific embodiment, the compound of formula I is compound 1.

The compound of formula I or compound 1, as described herein, includes both the recited compounds as well as individual isomers and mixtures of isomers. In each instance, the compound of formula I includes the pharmaceutically acceptable salts, hydrates, and/or solvates of the recited compounds and any individual isomers or mixture of isomers thereof.

In other embodiments, the compound of formula I or compound 1 can be the (L)-malate salt. The malate salt of the compound of formula I and of compound 1 is disclosed in PCT/US2010/021194 and U.S. Patent Application Ser. No. 61/325,095, the entire contents of each of which are incorporated herein by reference.

In other embodiments, the compound of formula I can be malate salt.

In other embodiments, the compound of formula I can be the (D)-malate salt.

In other embodiments, the compound of formula I can be the (L)-malate salt.

In other embodiments, compound 1 can be the malate salt.

In other embodiments, compound 1 can be (D)-malate salt.

In other embodiments, compound 1 can be the (L)-malate salt.

In another embodiment, the malate salt is in the crystalline N−1 form of the (L) malate salt and/or the (D) malate salt of the compound 1 as disclosed in U.S. Patent Application Ser. No. 61/325,095. See also WO 2008/083319 for the properties of crystalline enantiomers, including the N−1 and/or the N−2 crystalline forms of the malate salt of compound 1. Methods of making and characterizing such forms are fully described in PCT/US10/21194, which is incorporated herein by reference in its entirety.

In one embodiment the compound of formula I or compound 1 is administered concurrently (at the same time) or sequentially (one after the other) with compound 2. In a further embodiment, compounds 1 and 2 are administered once daily. In a further embodiment, compounds 1 and 2 are administered with fasting (i.e., without eating) for approximately two hours before and 1 hour after administration. Compounds 1 and 2 are preferably administered with a glass of water (approximately 8 ounces or 240 mL).

In another embodiment, compound 1 or a pharmaceutically acceptable salt thereof is administered orally once daily as a tablet or capsule. In another embodiment, compound 2 as the acetate is administered orally once daily as a tablet.

In another embodiment, compound 1 is administered orally as its free base or malate salt as a capsule or tablet.

The amounts of Compounds 1 and 2 that are administered will vary. In one embodiment, 1000 mg of Compound 2 is administered as four 250 mg tablets. In another embodiment, the amount of Compound 2 acetate is 750 mg, which is administered as three 250 mg tablets. In another embodiment, the amount of Compound 2 acetate is 500 mg which is administered as two 250 mg tablets. In another embodiment, the amount of Compound 2 acetate is 250 mg, which is administered as one 250 mg tablet.

In these and other embodiments, compound 1 is administered orally once daily as its free base or as the malate salt as a capsule or tablet. In a further embodiment, compound 1 is administered as the L-malate salt. In a further embodiment:
up to and including 100 mg of compound 1 is administered;
up to and including 95 mg of compound 1 is administered;
up to and including 90 mg of compound 1 is administered;
up to and including 85 mg of compound 1 is administered;
up to and including 80 mg of compound 1 is administered;
up to and including 75 mg of compound 1 is administered;
up to and including 70 mg of compound 1 is administered;
up to and including 65 mg of compound 1 is administered;
up to and including 60 mg of compound 1 is administered;
up to and including 55 mg of compound 1 is administered;
up to and including 50 mg of compound 1 is administered;
up to and including 45 mg of compound 1 is administered;
up to and including 40 mg of compound 1 is administered;
up to and including 35 mg of compound 1 is administered;
up to and including 30 mg of compound 1 is administered;
up to and including 25 mg of compound 1 is administered;
up to and including 20 mg of compound 1 is administered;
up to and including 15 mg of compound 1 is administered;
up to and including 10 mg of compound 1 is administered; or
up to and including 5 mg of compound 1 is administered.

In these and other embodiments, up to and including 1000 mg of Compound 2 acetate is administered once daily with fasting in combination with Compound 1 which is administered orally once daily with fasting as its free base or as the malate salt as a capsule or tablet. In a further embodiment:
up to and including 100 mg of compound 1 is administered;
up to and including 95 mg of compound 1 is administered;
up to and including 90 mg of compound 1 is administered;
up to and including 85 mg of compound 1 is administered;
up to and including 80 mg of compound 1 is administered;
up to and including 75 mg of compound 1 is administered;
up to and including 70 mg of compound 1 is administered;
up to and including 65 mg of compound 1 is administered;
up to and including 60 mg of compound 1 is administered;
up to and including 55 mg of compound 1 is administered;
up to and including 50 mg of compound 1 is administered;
up to and including 45 mg of compound 1 is administered;
up to and including 40 mg of compound 1 is administered;
up to and including 35 mg of compound 1 is administered;
up to and including 30 mg of compound 1 is administered;
up to and including 25 mg of compound 1 is administered;
up to and including 20 mg of compound 1 is administered;
up to and including 15 mg of compound 1 is administered;
up to and including 10 mg of compound 1 is administered; or
up to and including 5 mg of compound 1 is administered.

In these and other embodiments, up to and including 750 mg of Compound 2 acetate is administered once daily with fasting in combination with Compound 1 which is administered orally once daily with fasting as its free base or as the malate salt as a capsule or tablet. In a further embodiment:
up to and including 100 mg of compound 1 is administered;
up to and including 95 mg of compound 1 is administered;
up to and including 90 mg of compound 1 is administered;
up to and including 85 mg of compound 1 is administered;
up to and including 80 mg of compound 1 is administered;
up to and including 75 mg of compound 1 is administered;
up to and including 70 mg of compound 1 is administered;
up to and including 65 mg of compound 1 is administered;
up to and including 60 mg of compound 1 is administered;
up to and including 55 mg of compound 1 is administered;
up to and including 50 mg of compound 1 is administered;
up to and including 45 mg of compound 1 is administered;
up to and including 40 mg of compound 1 is administered;
up to and including 35 mg of compound 1 is administered;
up to and including 30 mg of compound 1 is administered;
up to and including 25 mg of compound 1 is administered;
up to and including 20 mg of compound 1 is administered;
up to and including 15 mg of compound 1 is administered;
up to and including 10 mg of compound 1 is administered; or
up to and including 5 mg of compound 1 is administered.

In these and other embodiments, up to and including 500 mg of Compound 2 acetate is administered once daily with fasting in combination with Compound 1 which is administered orally once daily with fasting as its free base or as the malate salt as a capsule or tablet containing:
up to and including 100 mg of compound 1;
up to and including 95 mg of compound 1;
up to and including 90 mg of compound 1;
up to and including 85 mg of compound 1;
up to and including 80 mg of compound 1;
up to and including 75 mg of compound 1;
up to and including 70 mg of compound 1;
up to and including 65 mg of compound 1;
up to and including 60 mg of compound 1;
up to and including 55 mg of compound 1;
up to and including 50 mg of compound 1;
up to and including 45 mg of compound 1;
up to and including 40 mg of compound 1;
up to and including 35 mg of compound 1;
up to and including 30 mg of compound 1;
up to and including 25 mg of compound 1;
up to and including 20 mg of compound 1;
up to and including 15 mg of compound 1;
up to and including 10 mg of compound 1; or
up to and including 5 mg of compound 1.

In these and other embodiments, up to and including 250 mg of Compound 2 acetate is administered once daily with fasting in combination with Compound 1 which is administered orally once daily with fasting as its free base or as the malate salt as a capsule or tablet. In a further embodiment:
up to and including 100 mg of compound 1 is administered;
up to and including 95 mg of compound 1 is administered;
up to and including 90 mg of compound 1 is administered;
up to and including 85 mg of compound 1 is administered;
up to and including 80 mg of compound 1 is administered;
up to and including 75 mg of compound 1 is administered;
up to and including 70 mg of compound 1 is administered;
up to and including 65 mg of compound 1 is administered;
up to and including 60 mg of compound 1 is administered;
up to and including 55 mg of compound 1 is administered;
up to and including 50 mg of compound 1 is administered;
up to and including 45 mg of compound 1 is administered;
up to and including 40 mg of compound 1 is administered;
up to and including 35 mg of compound 1 is administered;
up to and including 30 mg of compound 1 is administered;
up to and including 25 mg of compound 1 is administered;
up to and including 20 mg of compound 1 is administered;
up to and including 15 mg of compound 1 is administered;
up to and including 10 mg of compound 1 is administered; or
up to and including 5 mg of compound 1 is administered.

In other embodiments, 1000 mg of Compound 2 acetate is administered once daily with fasting in combination with Compound 1 as a tablet or capsule formulation containing 60, 40, or 20 mg of Compound 1 which is administered orally once daily with fasting as its free base or as the malate salt.

In other embodiments, 750 mg of Compound 2 acetate is administered once daily with fasting in combination with is administered once daily with fasting in combination with a Compound 1 as a tablet or capsule formulation containing 60, 40, or 20 mg of Compound 1 which is administered orally once daily with fasting as its free base or as the malate salt.

In other embodiments, 500 mg of Compound 2 acetate is administered once daily with fasting in combination with is administered once daily with fasting in combination with a Compound 1 as a tablet or capsule formulation containing 60, 40, or 20 mg of Compound 1 which is administered orally once daily with fasting as its free base or as the malate salt.

In other embodiments, 250 mg of Compound 2 acetate is administered once daily with fasting in combination with is administered once daily with fasting in combination with a Compound 1 as a tablet or capsule formulation containing 60, 40, or 20 mg of Compound 1 which is administered orally once daily with fasting as its free base or as the malate salt.

In these and other embodiments, prednisone or prednisolone is optionally administered as part of the combination. In a preferred embodiment, prednisone is optionally administered as part of the combination. In one embodiment, 5 mg of prednisone is administered twice daily to a patient undergoing the treatment.

In another embodiment, compound 1 is administered as its free base or malate salt orally once daily as a tablet as provided in the following table.

| Ingredient | (% w/w) |
|---|---|
| Compound 1 | 31.68 |
| Microcrystalline Cellulose | 38.85 |
| Lactose anhydrous | 19.42 |
| Hydroxypropyl Cellulose | 3.00 |
| Croscarmellose Sodium | 3.00 |
| Total Intra-granular | 95.95 |
| Silicon dioxide, Colloidal | 0.30 |
| Croscarmellose Sodium | 3.00 |
| Magnesium Stearate | 0.75 |
| | 100.00 |

In another embodiment, compound 1 is administered orally as its free base or malate salt once daily as a tablet as provided in the following table.

| Ingredient | (% w/w) |
|---|---|
| Compound 1 | 25.0-33.3 |
| Microcrystalline Cellulose | q.s |
| Hydroxypropyl Cellulose | 3 |
| Poloxamer | 0-3 |
| Croscarmellose Sodium | 6.0 |
| Colloidal Silicon Dioxide | 0.5 |
| Magnesium Stearate | 0.5-1.0 |
| | 100 |

In another embodiment, compound 1 is administered orally as its free base or malate salt once daily as a tablet as provided in the following table.

| Ingredient | Theoretical Quantity (mg/unit dose) |
|---|---|
| Compound 1 | 100.0 |
| Microcrystalline Cellulose PH-102 | 155.4 |
| Lactose Anhydrous 60M | 77.7 |
| Hydroxypropyl Cellulose, EXF | 12.0 |
| Croscarmellose Sodium | 24 |
| Colloidal Silicon Dioxide | 1.2 |
| Magnesium Stearate (Non-Bovine) | 3.0 |
| Opadry Yellow | 16.0 |
| Total | 416 |

In another embodiment, compound 1 is administered orally as its free base or malate salt once daily as a tablet as provided in the following table.

| Ingredient | Function | % w/w |
|---|---|---|
| Cabozantinib Drug Substance (25% drug load as | Active | 31.7 |
| free base) | Ingredient | |
| Microcrystalline Cellulose (Avicel PH-102) | Filler | 38.9 |
| Lactose Anhydrous (60M) | Filler | 19.4 |
| Hydroxypropyl Cellulose (EXF) | Binder | 3.0 |
| Croscarmellose Sodium (Ac-Di-Sol) | Disenegrant | 6.0 |
| Colloidal Silicon Dioxide, | Glidant | 0.3 |
| Magnesium Stearate | Lubricant | 0.75 |
| Opadry Yellow Film Coating which includes: | | |
| HPMC 2910/Hypromellose 6 cp | | |
| Titanium dioxide | Film Coating | 4.00 |
| Triacetin | | |
| Iron Oxide Yellow | | |

Compound 2 is administered as the acetate as abiraterone acetate 250-mg tablets. The tablets are oval shaped and white to off-white in color. The tablets contain abiraterone acetate and compendial (USP/NF/EP) grade lactose monohydrate, microcrystalline cellulose, croscarmellose sodium, povidone, sodium lauryl sulfate, magnesium stearate, colloidal silicon dioxide, and purified water.

Any of the tablet formulations provided above can be adjusted according to the dose of compound 1 desired. Thus, the amount of each of the formulation ingredients can be proportionally adjusted to provide a tablet formulation containing various amounts of compound 1 as provided in the previous paragraphs. In another embodiment, the formulations can contain 20, 40, 60, or 80 mg of compound 1.

The antitumor effect of the combination of the invention is measured using serological and radiographic methods available to the skilled practitioner. With respect to serological methods, patients will be required to have a rising prostate specific antigen, hereinafter PSA. PSA levels will be assessed before the study then every 4 weeks thereafter. Patients will be evaluated for serological response from the time of their first treatment with therapy if a detectable PSA at commencement of the study. Patients will be assigned a PSA response according to the following criteria:
Complete Serological Response: PSA level less than 0.2 ng/mL measured for 2 consecutive measurements at least 4 weeks apart.
Serological Partial Response (PR): Decline of PSA value, referenced to the pre-study level, by greater than or equal to 50% for 2 consecutive measurements at least 2 weeks apart.
PSA Stable Disease: Patients who do not meet the criteria for response (CR or PR) or serological progression.
Serological Progression (PD): Observed when the PSA demonstrates an increase that is more than 50% of nadir, taking as reference the lowest recorded PSA level since starting therapy. Two consecutive increases must be documented with each measurement obtained at least 2 weeks apart. On occasions, there may be an intermediate fluctuant value. In accordance with the Recommendations of the Prostate Cancer Clinical Trials Working Group, this will not restart the evaluation period so long as the intermediate value was not below the previous nadir. The date of first recorded increase (not defeated by a subsequent drop in PSA level to create a new nadir) will be deemed the date of progression. If a patient achieves a PSA that is less than 2 ng/mL, progression will only be deemed to have been confirmed once: (1) There has been an observed rise that is more than 50% of nadir since starting ADT; AND (2) The confirming increase was to a value that is more than 2.0 ng/mL (the unconfirmed and second increase may be a value that is less than 2.0 ng/mL but greater than 50% of nadir since starting ADT).

In one embodiment, a complete serological response is observed in patients being treated with the combination. In another embodiment, a serological partial response is observed in patients being treated with the combination. In a further embodiment, stable disease is observed in patients being treated with the combination.

With respect to radiographic methods, radiographic disease progression is defined by RECIST 1.1 for soft tissue disease, or the appearance of two or more new bone lesions on bone scan. Progression in the absence of clear symptomatic worsening at the first scheduled reassessment at Week 8 requires a confirmatory scan 6 or more weeks later. Standard imaging procedures available to the skilled practitioner, including technetium bone scans and CT scans can be used to measure radiographic effect. Other radiographic methods such as NaF and FDG-PET may also be used to measure radiographic effect.

### Embodiments

The invention is further defined by the following non-limiting embodiments.

### Embodiment 1

A method of treating cancer, comprising administering a patient in need of such treatment a compound of formula I:

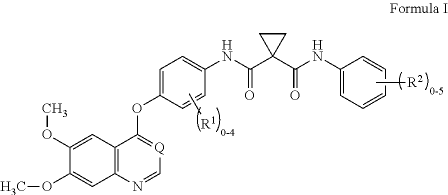

or a pharmaceutically acceptable salt thereof or a pharmaceutical composition comprising the compound of formula I and a pharmaceutically acceptable carrier, wherein:

R¹ is halo;

R² is halo; and

Q is CH or N;

in combination with a compound 2:

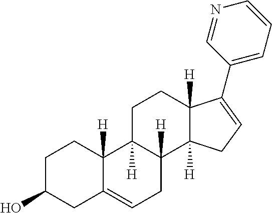

or a pharmaceutically acceptable salt thereof or a pharmaceutical composition comprising the compound of formula II and a pharmaceutically acceptable carrier.

### Embodiment 2

The method of embodiment 1, wherein the compound of formula I is compound 1.

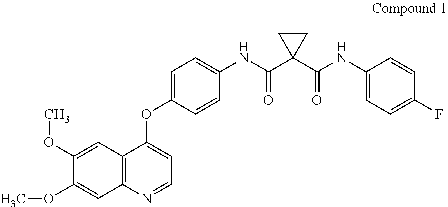 [SMILES: COc1cc2nccc(Oc3ccc(NC(=O)C4(C(=O)Nc5ccc(F)cc5)CC4)cc3)c2cc1OC]

### Embodiment 3

The method of embodiments 1-2, wherein compound 1 is administered as the L-malate salt.

### Embodiment 4

The method of embodiments 1-3 wherein compound 2 is administered as the acetate.

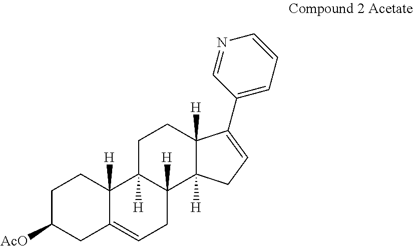

### Embodiment 5

The method of embodiments 1-4, wherein the cancer is castration resistant prostate cancer.

### Embodiment 6

The method of embodiments 1-5, wherein Compound 1 and compound 2 are administered concurrently or sequentially.

### Embodiment 7

The method of embodiments 1-6, wherein up to and including 1000 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

### Embodiment 8

The method of embodiments 1-7, wherein up to and including 1000 mg of Compound 2 is administered to the patient once daily with fasting in combination with 60 mg, 40 mg, or 20 mg of compound 1 once daily with fasting.

### Embodiment 9

The method of embodiments 1-8, wherein up to and including 750 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

### Embodiment 10

The method of embodiments 1-9, wherein up to 750 mg of Compound 2 is administered to the patient once daily with fasting in combination with 60 mg, 40 mg, or 20 mg of compound 1 once daily with fasting.

### Embodiment 11

The method of embodiments 1-10, wherein up to and including 500 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

### Embodiment 12

The method of embodiments 1-11, wherein up to and including 500 mg of Compound 2 is administered to the patient once daily with fasting in combination with 60 mg, 40 mg, or 20 mg of compound 1 once daily with fasting.

### Embodiment 13

The method of embodiments 1-12, wherein up to and including 250 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

### Embodiment 14

The method of embodiments 1-13, wherein up to and including 250 mg of Compound 2 is administered to the patient once daily with fasting in combination with 60 mg, 40 mg, or 20 mg of compound 1 once daily with fasting.

### Embodiment 15

The method of embodiments 1-14, further comprising prednisone or prednisolone.

### Embodiment 16

The method of embodiments 1-15, further comprising 5 mg prednisone administered twice daily.

### Embodiment 17

The method of embodiments 1-16, wherein a complete serological response is observed in patients being treated with the combination.

### Embodiment 18

The method of embodiments 1-17, wherein a serological partial response is observed in patients being treated with the combination.

### Embodiment 19

The method of embodiments 1-18, wherein stable disease is observed in patients being treated with the combination.

### Preparation of Compound 1

### Preparation of 1-(4-Fluorophenylcarbamoyl)cyclopropanecarboxylic acid (Compound A-1)

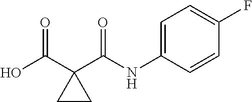 [SMILES: O=C(O)C1(C(=O)Nc2ccc(F)cc2)CC1]

The starting 1,1-cyclopropanedicarboxylic acid was treated with thionyl chloride (1.05 equivalents) in approximately 8 volumes of isopropyl acetate at 25° C. for 5 hours. The resulting mixture was then treated with a solution of 4-fluoroaniline (1.1 equivalents) and triethylamine (1.1 equivalents) in isopropyl acetate (2 volumes) over 1 hour. The product slurry was quenched with 5N NaOH solution (5 volumes), and the aqueous phase was discarded. The organic phase was extracted with 0.5N NaOH solution (10 volumes), and the basic extract was washed with heptane (5 volumes) and subsequently acidified with 30% HCl solution to give a slurry. Compound A-1 was isolated by filtration.

Compound A-1 was prepared on a 1.00 kg scale using 1,1-cyclopropanedicarboxylic acid as the limiting reagent to furnish 1.32 kg of Compound A-1 (77% isolated yield; 84% mass balance) with 99.92% purity (HPLC) and 100.3% assay.

### Preparation of N-(4-{[6,7-bis(methyloxy)quinolin-4-yl]oxy}phenyl)-N′-(4-fluorophenyl)cyclopropane-1,1-dicarboxamide (Compound 1) and the (L)-malate salt thereof

A synthetic route that can be used for the preparation of N-(4-{[6,7-bis(methyloxy)quinolin-4-yl]oxy}phenyl)-N′-(4-fluorophenyl)cyclopropane-1,1-dicarboxamide and the (L)-malate salt thereof is depicted in Scheme 1.

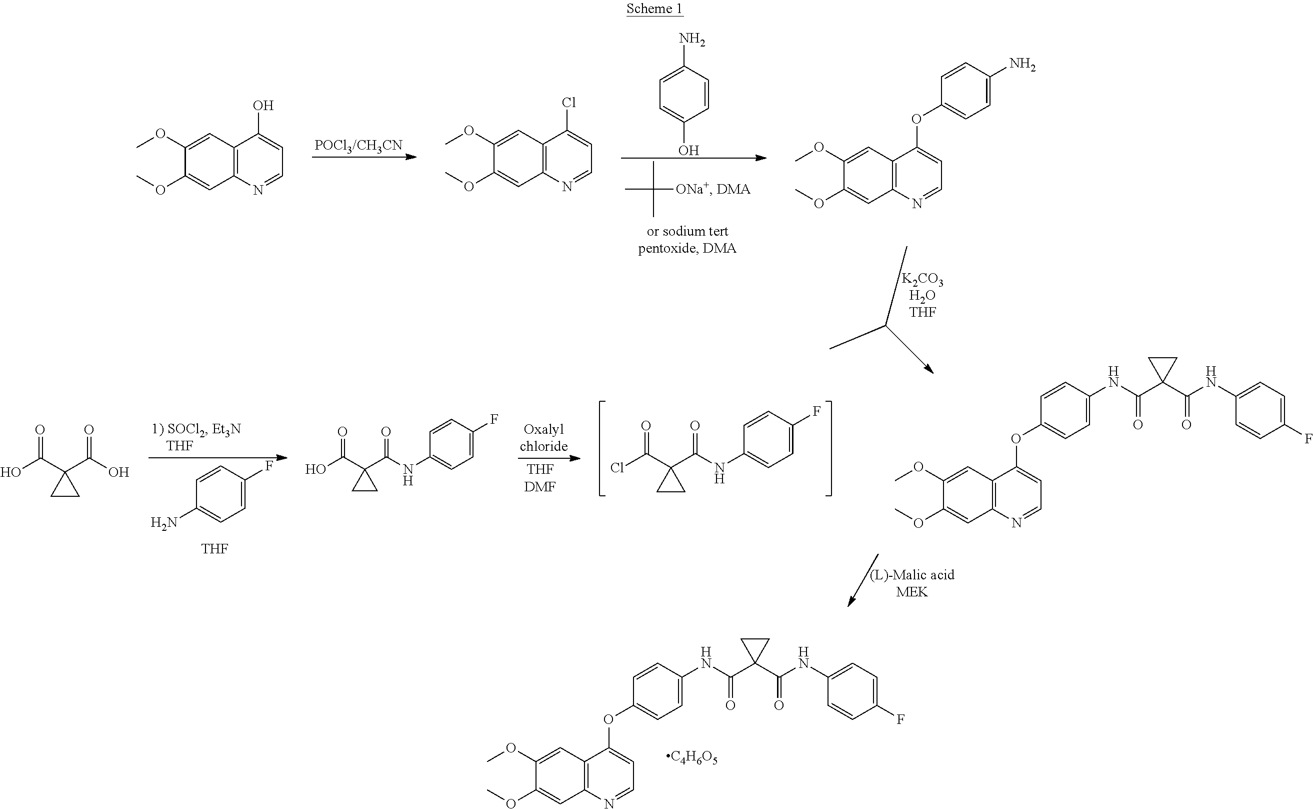

Another synthetic route that can be used for the preparation of N-(4-{[6,7-bis(methyloxy)quinolin-4-yl]oxy}phenyl)-N′-(4-fluorophenyl)cyclopropane-1,1-dicarboxamide and the (L)-malate salt thereof is depicted in Scheme 2.

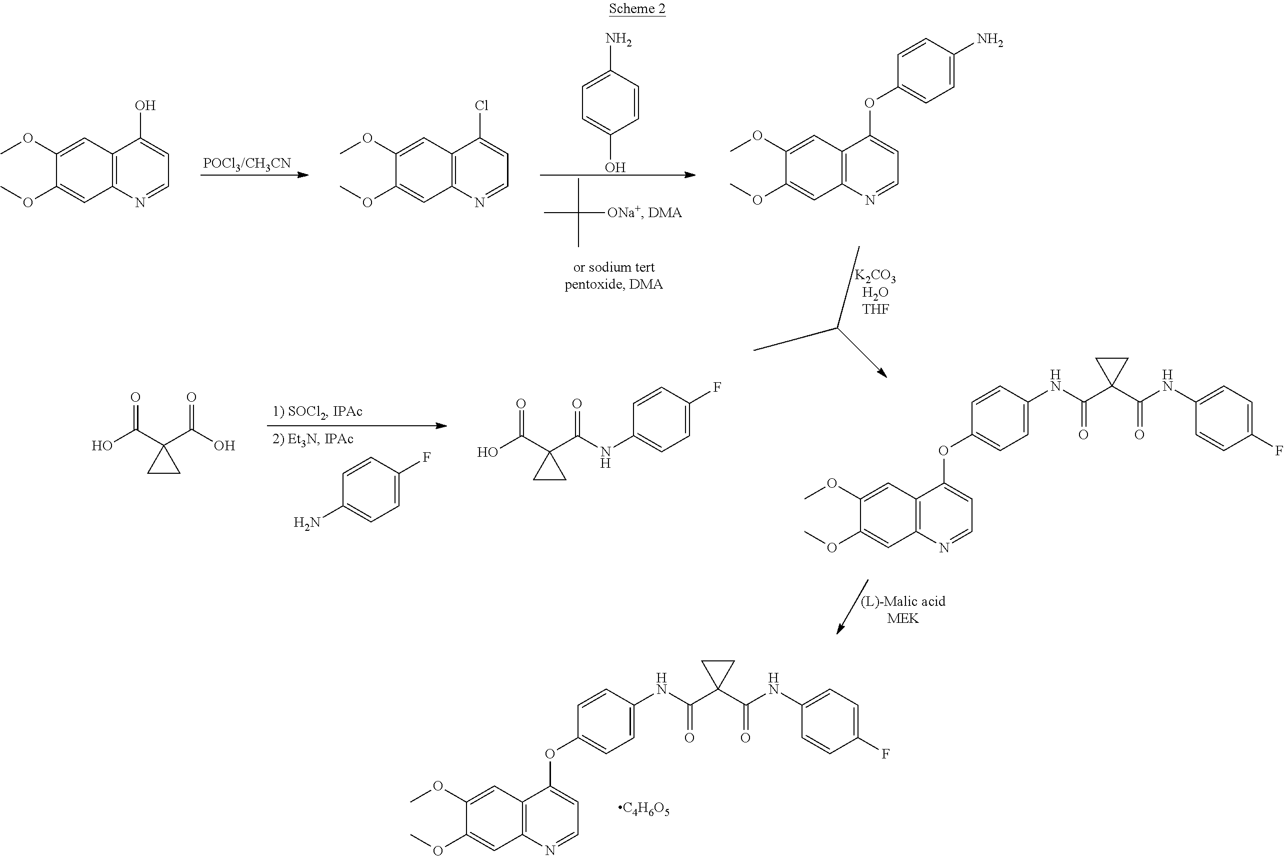

### Preparation of 4-Chloro-6,7-dimethoxy-quinoline

A reactor was charged sequentially with 6,7-dimethoxy-quinoline-4-ol (47.0 kg) and acetonitrile (318.8 kg). The resulting mixture was heated to approximately 60° C., and phosphorus oxychloride (POCl₃, 130.6 kg) was added. After the addition of POCl₃, the temperature of the reaction mixture was raised to approximately 77° C. The reaction was deemed complete (approximately 13 hours) when less than 3% of the starting material remained, as measured by in-process high-performance liquid chromatography [HPLC] analysis. The reaction mixture was cooled to approximately 2 to 7° C. and then quenched into a chilled solution of dichloromethane (DCM, 482.8 kg), 26% NH₄OH (251.3 kg), and water (900 L). The resulting mixture was warmed to approximately 20 to 25° C., and phases were separated. The organic phase was filtered through a bed of AW hyflo super-cel NF (Celite; 5.4 kg), and the filter bed was washed with DCM (118.9 kg). The combined organic phase was washed with brine (282.9 kg) and mixed with water (120 L). The phases were separated, and the organic phase was concentrated by vacuum distillation with the removal of solvent (approximately 95 L residual volume). DCM (686.5 kg) was charged to the reactor containing organic phase and concentrated by vacuum distillation with the removal of solvent (approximately 90 L residual volume). Methyl t-butyl ether (MTBE, 226.0 kg) was then charged, and the temperature of the mixture was adjusted to −20 to −25° C. and held for 2.5 hours resulting in solid precipitate, which was then filtered, washed with n-heptane (92.0 kg), and dried on a filter at approximately 25° C. under nitrogen to afford the title compound (35.6 kg).

### Preparation of 4-(6, 7-Dimethoxy-quinoline-4-yloxy)-phenylamine

4-Aminophenol (24.4 kg) dissolved in N,N-dimethylacetamide (DMA, 184.3 kg) was charged to a reactor containing 4-chloro-6,7-dimethoxyquinoline (35.3 kg), sodium t-butoxide (21.4 kg), and DMA (167.2 kg) at 20-25° C. This mixture was then heated to 100-105° C. for approximately 13 hours. After the reaction was deemed complete as determined using in-process HPLC analysis (less than 2% starting material remaining), the reactor contents were cooled at 15 to 20° C., and water (pre-cooled, 2 to 7° C., 587 L) was charged at a rate to maintain 15 to 30° C. temperature. The resulting solid precipitate was filtered, washed with a mixture of water (47 L) and DMA (89.1 kg), and finally washed with water (214 L). The filter cake was then dried at approximately 25° C. on filter to yield crude 4-(6,7-dimethoxy-quinoline-4-yloxy)-phenylamine (59.4 kg wet, 41.6 kg dry calculated based on limit of detection, hereinafter “LOD”). Crude 4-(6,7-dimethoxy-quinoline-4-yloxy)-phenylamine was refluxed (approximately 75° C.) in a mixture of tetrahydrofuran (THF, 211.4 kg) and DMA (108.8 kg) for approximately 1 hour, then cooled to 0 to 5° C., and aged for approximately 1 hour, after which time the solid was filtered, washed with THF (147.6 kg), and dried on a filter under vacuum at approximately 25° C. to yield 4-(6,7-dimethoxy-quinoline-4-yloxy)-phenylamine (34.0 kg).

### Alternative Preparation of 4-(6,7-Dimethoxy-quinoline-4-yloxy)-phenylamine

4-chloro-6,7-dimethoxyquinoline (34.8 kg), 4-Aminophenol (30.8 kg), and sodium tert-pentoxide (1.8 equivalents) 88.7 kg, 35 weight percent in THF) were charged to a reactor, followed by N,N-dimethylacetamide (DMA, 293.3 kg). This mixture was then heated to 105 to 115° C. for approximately 9 hours. After the reaction was deemed complete as determined using in-process HPLC analysis (less than 2% starting material remaining), the reactor contents were cooled at 15 to 25° C., and water (315 kg) was added over a two hour period while maintaining the temperature between 20 and 30° C. The reaction mixture was then agitated for an additional hour at 20 to 25° C. The crude product was collected by filtration and washed with a mixture of 88 kg water and 82.1 kg DMA, followed by 175 kg water. The product was dried on a filter drier for 53 hours. The LOD showed less than 1% w/w.

In an alternative procedure, 1.6 equivalents of sodium tert-pentoxide were used, and the reaction temperature was increased from 110 to 120° C. In addition, the cool down temperature was increased to 35 to 40° C., and the starting temperature of the water addition was adjusted to 35 to 40° C., with an allowed exotherm to 45° C.

### Preparation of 1-(4-Fluoro-phenylcarbamoyl)-cyclopropanecarbonyl chloride

Oxalyl chloride (12.6 kg) was added to a solution of 1-(4-fluoro-phenylcarbamoyl)-cyclopropanecarboxylic acid (22.8 kg) in a mixture of THF (96.1 kg) and N, N-dimethylformamide (DMF; 0.23 kg) at a rate such that the batch temperature did not exceed 25° C. This solution was used in the next step without further processing.

### Alternative Preparation of 1-(4-Fluoro-phenylcarbamoyl)-cyclopropanecarbonyl chloride

A reactor was charged with 1-(4-fluoro-phenylcarbamoyl)-cyclopropanecarboxylic acid (35 kg), DMF (344 g), and THF (175 kg). The reaction mixture was adjusted to 12 to 17° C., and then to the reaction mixture was charged 19.9 kg of oxalyl chloride over a period of 1 hour. The reaction mixture was left stirring at 12 to 17° C. for 3 to 8 hours. This solution was used in the next step without further processing.

### Preparation of cyclopropane-1,1-dicarboxylic acid [4-(6,7-dimethoxy-quinoline-4-yloxy)-phenyl]-amide(4-fluoro-phenyl)-amide

The solution from the previous step containing 1-(4-fluoro-phenylcarbamoyl)-cyclopropanecarbonyl chloride was added to a mixture of compound 4-(6,7-dimethoxy-quinoline-4-yloxy)-phenylamine (23.5 kg) and potassium carbonate (31.9 kg) in THF (245.7 kg) and water (116 L) at a rate such that the batch temperature did not exceed 30° C. When the reaction was complete (in approximately 20 minutes), water (653 L) was added. The mixture was stirred at 20 to 25° C. for approximately 10 hours, which resulted in the precipitation of the product. The product was recovered by filtration, washed with a pre-made solution of THF (68.6 kg) and water (256 L), and dried first on a filter under nitrogen at approximately 25° C. and then at approximately 45° C. under vacuum to afford the title compound (41.0 kg, 38.1 kg, calculated based on LOD).

### Alternative Preparation of cyclopropane-1,1-dicarboxylic acid [4-(6,7-dimethoxy-quinoline-4-yloxy)-phenyl]-amide(4-fluoro-phenyl)-amide

A reactor was charged with 4-(6,7-dimethoxy-quinoline-4-yloxy)-phenylamine (35.7 kg, 1 equivalent), followed by THF (412.9 kg). To the reaction mixture was charged a solution of K₂CO₃ (48.3 kg) in water (169 kg). The acid chloride solution of described in the Alternative Preparation of 1-(4-Fluoro-phenylcarbamoyl)-cyclopropanecarbonyl chloride above was transferred to the reactor containing 4-(6,7-dimethoxy-quinoline-4-yloxy)-phenylamine while maintaining the temperature between 20 to 30° C. over a minimum of two hours. The reaction mixture was stirred at 20 to 25° C. for a minimum of three hours. The reaction temperature was then adjusted to 30 to 25° C., and the mixture was agitated. The agitation was stopped, and the phases of the mixture were allowed to separate. The lower aqueous phase was removed and discarded. To the remaining upper organic phase was added water (804 kg). The reaction was left stirring at 15 to 25° C. for a minimum of 16 hours.

The product precipitated and was filtered and washed with a mixture of water (179 kg) and THF (157.9 kg) in two portions. The crude product was dried under a vacuum for at least two hours. The dried product was then taken up in THF (285.1 kg). The resulting suspension was transferred to reaction vessel and agitated until the suspension became a clear (dissolved) solution, which required heating to 30 to 35° C. for approximately 30 minutes. Water (456 kg) was then added to the solution, as well as SDAG-1 ethanol (20 kg, ethanol denatured with methanol over two hours). The mixture was agitated at 15 to 25° C. for at least 16 hours. The product was filtered and washed with a mixture of water (143 kg and 126.7 kg THF (143 kg) in two portions. The product was dried at a maximum temperature set point of 40° C.

In an alternative procedure, the reaction temperature during acid chloride formation was adjusted to 10 to 15° C. The recrystallization temperature was changed from 15 to 25° C. to 45 to 50° C. for 1 hour and then cooled to 15 to 25° C. over 2 hours.

### Preparation of cyclopropane-1,1-dicarboxylic acid [4-(6,7-dimethoxy-quinoline-4-yloxy)-phenyl]-amide(4-fluoro-phenyl)-amide, cabozantinib (L) malate salt

Cyclopropane-1,1-dicarboxylic acid [4-(6,7-dimethoxy-quinoline-4-yloxy)-phenyl]-amide(4-fluoro-phenyl)-amide (13.3 kg), L-malic acid (4.96 kg), methyl ethyl ketone (MEK; 188.6 kg) and water (37.3 kg) were charged to a reactor, and the mixture was heated to reflux (approximately 74° C.) for approximately 2 hours. The reactor temperature was reduced to 50 to 55° C., and the reactor contents were filtered. These sequential steps described above were repeated two more times starting with similar amounts of cyclopropane-1,1-dicarboxylic acid [4-(6,7-dimethoxy-quinoline-4-yloxy)-phenyl]-amide(4-fluoro-phenyl)-amide (13.3 kg), L-Malic acid (4.96 kg), MEK (198.6 kg), and water (37.2 kg). The combined filtrate was azeotropically dried at atmospheric pressure using MEK (1133.2 kg) (approximate residual volume 711 L; KF<0.5% w/w) at approximately 74° C. The temperature of the reactor contents was reduced to 20 to 25° C. and held for approximately 4 hours, resulting in solid precipitate which was filtered, washed with MEK (448 kg), and dried under vacuum at 50° C. to afford the title compound (45.5 kg).

### Alternative Preparation of cyclopropane-1,1-dicarboxylic acid [4-(6,7-dimethoxy-quinoline-4-yloxy)-phenyl]-amide(4-fluoro-phenyl)-amide, (L) malate salt

Cyclopropane-1,1-dicarboxylic acid [4-(6,7-dimethoxy-quinoline-4-yloxy)-phenyl]-amide(4-fluoro-phenyl)-amide (47.9 kg), L-malic acid (17.2 kg), methyl ethyl ketone (658.2 kg), and water (129.1 kg) were charged to a reactor, and the mixture was heated 50 to 55° C. for approximately 1 to 3 hours and then at 55 to 60° C. for an additional 4 to 5 hours. The mixture was clarified by filtration through a 1 μm cartridge. The reactor temperature was adjusted to 20 to 25° C. and vacuum distilled with a vacuum at 150 to 200 mm Hg with a maximum jacket temperature of 55° C. to the volume range of 558 to 731 L.

The vacuum distillation was performed two more times with the charge of 380 kg and 380.2 kg methyl ethyl ketone, respectively. After the third distillation, the volume of the batch was adjusted to 18 v/w of Cyclopropane-1,1-dicarboxylic acid [4-(6,7-dimethoxy-quinoline-4-yloxy)-phenyl]-amide(4-fluoro-phenyl)-amide by charging methyl ethyl ketone (159.9 kg) to give a total volume of 880 L. An additional vacuum distillation was carried out by adjusting methyl ethyl ketone (245.7 kg). The reaction mixture was left with moderate agitation at 20 to 25° C. for at least 24 hours. The product was filtered and washed with methyl ethyl ketone (415.1 kg) in three portions. The product was dried under a vacuum with the jacket temperature set point at 45° C.

In an alternative procedure, the order of addition was changed so that a solution of L-malic acid (17.7 kg) dissolved in water (129.9 kg) was added to Cyclopropane-1,1-dicarboxylic acid [4-(6,7-dimethoxy-quinoline-4-yloxy)-phenyl]-amide(4-fluoro-phenyl)-amide (48.7 kg) in methyl ethyl ketone (673.3 kg).

### Example 1

### Phase I Study of Cabozantinib with Abiraterone for the Treatment of Castration Resistant Prostate Cancer

### Study Description

This is a phase I dosing finding study of cabozantinib with co-administration of abiraterone at the full dose of 1000 mg per day and 5 mg twice a day of prednisone. There will be up to three expansion cohorts to further evaluate the toxicity profile and antitumor activities of the combination therapies.

The study will be conducted in 2 parts. Part A is phase 1 dose-escalation part to establish the maximum tolerated dose (hereinafter “MTD”) of cabozantinib in combination with abiraterone. Part B is a dose expansion part including up to 3 dose levels which will have been determined to be safe and tolerable in Part A. The cohorts may be expanded to a maximum of 12 subjects at each dose level (including the subjects from Part A).

Part A is a 3+3 open-label, dose-escalation component. A standard “3 plus 3” dose-escalation design will be used. Subjects will be assigned to receive abiraterone at the labeled-dose of 1000 mg per day. Cabozantinib will also be given once daily. The starting doses of cabozantinib will be as follows:

| Dose Level | Cabozantinib Dose |
|---|---|
| 1 | 20 mg |
| 2 | 40 mg |
| 3 | 60 mg |

Three subjects will initially be accrued at a dose level, starting with dose level 1. If 0 of 3 subjects have a dose-limiting toxicity (hereinafter “DLT”) in the first 4 weeks of treatment, dose escalation will proceed to the next dose level. If 1 subject of 3 experiences a DLT, an additional 3 subjects will be accrued at that dose level. If a total of 2 or more of the 6 subjects experience a DLT, this dose level will be considered to have exceeded the MTD. Doses of cabozantinib greater than 60 mg will not be evaluated. To be evaluable for DLT assessment, a patient must have received greater than 85% of planned dosing of both drugs in the first 4 weeks and toxicity is assessable. Any patient who stops therapy prior to 4 weeks for any reason other than a DLT will be replaced if they have not received greater than 85% of planned dosing. If patients did receive greater than 85% of planned dosing, they will be eligible for DLT assessment. The following table gives the probability of escalating to the next dose assuming various true but unknown dose limiting toxicity (DLT) rates.

| | DLT rate | | | | |
|---|---|---|---|---|---|
| | 0.1 | 0.2 | 0.3 | 0.4 | 0.5 |
| P(esc\|DLT rate) | 0.91 | 0.71 | 0.49 | 0.31 | 0.17 |

In order to better characterize the safety and preliminary antitumor activity of the dose levels, up to 3 dose levels of cabozantinib which have been determined to be safe and tolerable in Part A will be expanded in Part B. There will be up to 12 evaluable subjects (including patients from part A) in each expansion cohort. An evaluable subject is a patient who has completed all first 4 weeks of therapy and toxicity is assessable i.e., any patient who stops therapy prior to receiving 85% of planned dose in the first weeks for any reason other than a DLT will be replaced. Patients will also receive 1000 mg of abiraterone with 5 mg twice a day of prednisone. Patients will be sequentially assigned to expansion cohorts potentially corresponding to dose levels 1, 2, and 3.

The selection of the dose level(s) to be expanded will be based on all available safety data (including any adverse events, hereinafter “AE,” and dose modification data after the DLT period as part of the longer term safety profile) and preliminary antitumor data from Part A. The sponsor may decide to stop accrual to any of the expansion cohorts based on the accumulating safety and preliminary antitumor/pharmacodynamic data.

### Primary Objective

The primary objective is to define the maximum tolerated dose of cabozantinib in combination with abiraterone. The primary endpoint is the rate of dose limiting toxicity (DLT) in the first 4 weeks of therapy when abiraterone is combined with escalating doses of cabozantinib.

### Secondary Objective

The secondary objective is to define a dosing regimen of abiraterone and cabozantinib suitable for further evaluation based on long term toxicity and efficacy data. The secondary endpoints include the following:
incidence of DLT (defined above) at any time on protocol therapy and frequency of dose reductions;
steady state trough concentrations of abiraterone and cabozantinib when given in combination;
quantitative impact of treatment on Technetium-99m-MDP bone scans as determined by MedQiA (% change of the positive area on a bone scan computed using semi-automated proprietary software);
quantitative impact of treatment on bone turnover measured by 18F sodium fluoride (NaF) PET scans (percent change in standardized uptake value (SUV));
time to radiographic progression or symptomatic deterioration from progressive CRPC;
time to skeletal related event;
time to treatment failure (progression or discontinue therapy due to toxicity) and overall survival of patients;
changes in blood borne markers of bone turnover and bone microenvironment (% change in markers compared with baseline);
PSA decline or rise on therapy as represented by a waterfall plot (% change compared with baseline);
changes in Circulating Tumor Cells (CTC) count before and after treatment (absolute numbers per 7.5 mL blood);
changes in soft tissue disease (if present) using CT imaging (% change in sum of longest dimension per RECIST); and
changes in soft tissue disease using FDG-PET (% change in SUV).

### Justification of Study Design

Cabozantinib has been associated with bone scan improvements independent of PSA declines (discordance) whereas abiraterone does cause PSA declines, as one would expect with a hormone manipulation. Radiographic and symptomatic progression are objective endpoints that are relevant for both drugs. Bone scan progression is the development of symptomatic lesions or appearance of two new lesions on imaging. Cabozantinib has been shown to decrease uptake on bone scans and, in some cases, to normalize them.

MedQIA, a radiology support company, has developed an algorithm to quantify changes in bone scan and accurately detail intrapatient changes.

When designing a definitive comparative trial, the feasibility of combination therapy needs to be reviewed in the light of long term tolerability to see if it is viable to dose the two agents together with the goal of determining whether the total duration of cancer control with the combination exceeds the time period covered by time to progression when the agents are given sequentially. In this case, it is possible both cabozantinib and abiraterone have median duration of cancer control for 9 months (18 month sequentially). However, to accomplish this assumes that all patients are suitable for treatment with the second agent at the time of progression (i.e., not progressed with deterioration in performance status or organ function). Therefore, a more realistic estimate of the cancer control/PFS for all patients starting potential sequential therapy is an increase of PFS by approximately 50 percent from 9 months to 14 months. It is therefore conceivable that the use of 2 active agents concurrently is more likely to achieve major cancer control and possibly achieve a median of 18 months disease control (i.e., 4 more months than the combination given sequentially). It is proposed that achievement of this degree of cancer control will make it worth translating the use of the combination into routine clinical practice.

Therefore, prior to commencement of a definitive comparative study of sequential versus concurrent cabozantinib and abiraterone trial, data detailing the optimal dose that can facilitate long term exposure is required. This is specifically relevant given the observation from existing studies that patients have been on cabozantinib monotherapy for many months (approximately up to 6 months in post-docetaxel in CRPC patients). A combination therapy may require dosing of both agents for more than 18 months. There are two potential strategies for choosing a dosing regimen for the combination arm: (i) Start at the MTD and dose reduce as needed if clinically significant toxicity emerges or (ii) start at a lower but still biologically active dose which has been shown to be more tolerable. As a result, after a dose escalation toxicity evaluation, this study will evaluate parallel arms of abiraterone at 1000 mg per day with the MTD of cabozantinib plus abiraterone combination and the lower biologically active dose(s) of cabozantinib with abiraterone. We will assess both toxicity/long term tolerability and measures of activity.

The study fixes the abiraterone dose at the clinically proven dose of 1000 mg, which has no major toxicities that preclude its long term use based on results from phase 3 trials. The dose of cabozantinib to be taken forward will be based on analysis of the composite of endpoints that considers both tolerability and efficacy (anti-cancer and pharmacodynamic activity). As such there are five scenarios; the first is that the lower dose arm is more effective and better tolerated due to patients having prolonged effective dosing. The second scenario is the lower dose arm is better tolerated but associated with less efficacy. The third scenario would be the lower dose arm has similar efficacy but better tolerability. The fourth scenario is the higher dose has better efficacy and more toxicity that is tolerable. The fifth scenario is the higher dose has greater efficacy but ongoing dosing requires frequent dose reductions. The final determination will be based on the totality of the data and weighting the clinical important variables (such as tolerability, long term cancer control, survival, definitive radiographic, or symptomatic progression evaluations) above laboratory values.

### Correlative Studies Background

The following correlative studies are planned and will be analyzed as secondary endpoints. This data will be used to help define the biologically active doses of cabozantinib in combination with abiraterone.

### Examination of Changes of Cancer Burden in Bone

Cabozantinib has been shown to result in notable improvements of technetium CT bone scans and, in some cases, has been shown to normalize the scan. It is presumed that because of the decrease (but not eradication) of cancer on CT imaging as well as the decrease in pain, this phenomenon is due to an effect both on the tumor compartment as well as the bone microenvironment. As such, the study will assess the impact the combination of abiraterone and escalating doses of cabozantinib has on bone turnover measured by quantitative technetium bone scan and the putatively more refined assessment with NaF PET. The study will also assess the impact of the combination on the soft tissue cancer component in the bone and extraosseous disease by FDG-PET and CT scans. Scans will be performed at baseline and after 8 weeks of therapy. The data from the baseline and 8 week readings and change from baseline to 8 weeks will be correlated with time to progression.

### Examination of Serum Drug Levels of Abiraterone and Cabozantinib at Steady State

Because of the potential for either drug to affect the metabolism of its companion, levels will be obtained at baseline and weekly for the first 4 weeks. Results for both abiraterone and cabozantinib will be grouped and analyzed by the cabozantinib drug dose at time of blood draw. For the first 4 weeks, patients will take the drugs at the same time in the morning on an empty stomach to ensure consistency. Patients will not take the drugs prior to pyruvate kinase (PK) blood draws which will be done in the morning in the clinic.

### Examination of Markers of Bone Turnover and Bone Microenvironment

As stated above, cabozantinib has been shown to have a profound effect on bone scans in some but not all patients. As such we will assess the impact the combination of abiraterone and escalating doses of cabozantinib on markers of bone turnover (some of which have been shown to be decreased on cabozantinib alone) as well as proteins of the bone microenvironment. Levels will be performed at baseline at week 4 and week 8 of therapy and at progression. The levels from baseline and 4 and 8 weeks, as well as change from baseline to week 4 and baseline to week 8 will be correlated with time to progression and with the effects on NaF PET and quantitative bone scan.

Markers of bone turnover with levels at baseline and at month 1 and 2 of therapy and at progression include:
Serum bone alkaline phosphatase,
Serum osteocalcin,
Serum osteopontin,
Serum osteonectin,
Serum scerlostin, and
Serum osteoprotegrin.

Cytokines and chemokines that support the cancer growth in the bone microenvironment either alone or in combination with abiraterone include:
TGFβ,
CCL-2,
RANK-ligand,
IL6, and
IL-8.

### Circulating Tumor Cells (CTCs)

Decrease in number of circulating tumor cells on abiraterone therapy has been shown to be associated with a longer overall survival. As such, we will quantify the number of CTCs at baseline and at 4 and 8 weeks of therapy when treated with the combination of abiraterone and escalating doses of cabozantinib. The levels from baseline and 4 and 8 weeks as well as change from baseline to week 4 and baseline to week 8 will be correlated with time to progression and effects on NaF PET and quantitative bone scan. Molecular interrogation of the CTCs will also be done (e.g., CPY17 levels by immunohistochemistry and AR variants by PCR) to assess for molecular changes which may emerge with abiraterone resistance.

### Participant Selection

### Inclusion Criteria:

The subject has a pathologically and radiologically confirmed, advanced, recurrent, or metastatic CRPC.
The subject must have CRPC, with serum testosterone less than 50 ng/dL.
The patient may be treatment-naïve, or have up to 2 prior chemotherapy regimens for CRPC. The therapy must include at least 225 mg/m² of docetaxel and docetaxel single agent followed by docetaxel plus carboplatin counts as two lines of therapy.
No prior lyase or CYP17A1 inhibitors (but prior ketoconazole allowed if at least 4 months prior to enrollment).
The subject must have discontinued flutamide and megestrol acetate at least 4 weeks, and bicalutamide or nilutamide at least 6 weeks before the first dose of study treatment and have documented a PSA rise after stopping the anti-androgen.
NOTE: Subjects must maintain a castrate state and, if not had an orchiectomy, must continue to receive LHRH or GnRH agonists.
Subjects enrolling to the study must have progressive disease (PD) on computerized tomography (CT), magnetic resonance imaging (MRI), or bone scan per mRECIST, by investigator assessment while on or within 4 months of docetaxel or cabazitaxel (if treated with cabaziataxel as well) based chemotherapy.
Progression of bone lesions must be diagnosed by bone scan, and unequivocal, i.e. not attributable to differences in scanning technique, change in imaging modality, or findings not representing tumor and according to PSAWG (Prostate Specific Antigen Working Group) criteria.
Progression by bone metastases with need for radiation therapy (documented by investigator) or need to change systemic therapy will qualify as progression to allow enrollment.
The subject has recovered to baseline or CTCAE (Common Terminology Criteria for Adverse Events)≦Grade 1 from toxicities related to prior treatment, except alopecia, lymphopenia, and other non-clinically significant AEs.
The subject is ≧18 years old on the day of consent.
The subject has an Eastern Cooperative Oncology Group (ECOG) performance status of 0 or 1.
The subject has organ and marrow function as follows:
Absolute neutrophil count (ANC)≧1500/mm3.
Platelets≧100,000/mm3.
Hemoglobin≧9 g/dL (can be post transfusion).
Total bilirubin≦1.5× the upper limit of normal. For subjects with known Gilbert's disease, bilirubin≦3.0 mg/dL.
Serum albumin≧2.8 g/dL.
Serum creatinine≦1.5× the upper limit of normal or calculated creatinine clearance≧60 mL/min or GFR (glomerular filtration rate)>40 ml/min. NOTE: For GFR estimation, the Cockcroft and Gault equation should be used: Male: GFR=CrCl (ml/min)=(140−age)×wt (kg)/(serum creatinine×72).
Serum potassium≧3.5.
Serum phosphorus>LLN.
Urine protein creatinine ratio (UPCR)≦1.
Lipase<1.5×ULN (upper limit of normal).
Alanine aminotransferase (ALT) and aspartate aminotransferase (AST)≦2.5× the institutional upper limit of normal if no liver involvement, or ≦5× the institutional upper limit of normal with liver involvement. (For patients with CRPC and documented bone metastases, AST can be >2.5×ULN if the investigator can provide evidence of no underlying liver dysfunction and the AST is originating from bone source).
Sexually active subjects must agree to use medically accepted barrier methods of contraception (e.g., condom) during the course of the study and for 4 months after the last dose of study drug(s), even if oral contraceptives are also used. All subjects of reproductive potential must agree to use both a barrier method and a second method of birth control.
The subject is able or willing to abide by the study protocol or cooperate fully with the investigator or designee.
The subject is capable of understanding and complying with the protocol requirements and has signed the informed consent document.

### Exclusion Criteria:

A subject who meets any of the following criteria is ineligible for the study:
The subject has received cytotoxic chemotherapy (including investigational cytotoxic chemotherapy) or biologic agents (e.g., cytokines or antibodies) within 3 weeks, or nitrosoureas/mitomycin C within 6 weeks before the first dose of study treatment.
Prior treatment with cabozantinib or other c-MET inhibitor.
Any prior therapy with a lyase inhibitor. In the case of ketoconazole, a patient can enroll if more than 120 days since last dose.
Concurrent use of hormonal therapies other than LHRH analogue or orchiectomy are not allowed (including anti-androgens, estrogens, 5 alpha reductase inhibitors or concurrent use of immunotherapy.
The subject has received radiation therapy:
   to the thoracic cavity or gastrointestinal tract within 3 months of the first dose of study treatment (Radiation to rib or thoracic vertebral metastases is allowed);
   to bone or brain metastasis within 14 days of the first dose of study treatment; or
   to any other site(s) within 28 days of the first dose of study treatment.
The subject has received radionuclide treatment within 6 weeks of the first dose of study treatment.
The subject has received prior treatment with a small molecule kinase inhibitor or a hormonal therapy (including investigational kinase inhibitors or hormones) within 14 days or five half-lives of the compound or active metabolites, whichever is longer, before the first dose of study treatment. Note: Subjects with prostate cancer currently receiving LHRH or GnRH agonists may be maintained on these agents.
The subject has received any other type of investigational agent within 28 days before the first dose of study treatment.
The subject has not recovered to baseline or CTCAE≦Grade 1 from toxicity due to all prior therapies except alopecia, lymphopenia, and other non-clinically significant AEs.
The subject has active brain metastases or epidural disease (Note: Subjects with brain metastases previously treated with whole brain radiation or radiosurgery or subjects with epidural disease previously treated with radiation or surgery who are asymptomatic and do not require steroid treatment for at least 2 weeks before starting study treatment are eligible. Neurosurgical resection of brain metastases or brain biopsy is permitted if completed at least 3 months before starting study treatment. Baseline brain scans are not required to confirm eligibility with patients with no history of brain metastases or symptoms concerning for CNS disease.)
The subject has prothrombin time (PT)/International Normalized Ratio (INR) or partial thromboplastin time (PTT) test results at screening≧1.3× the laboratory ULN.
The subject requires concomitant treatment, in therapeutic doses, with anticoagulants such as warfarin or warfarin-related agents, heparin, thrombin or FXa inhibitors, or antiplatelet agents (e.g., clopidogrel). Low dose aspirin (≦81 mg/day), low-dose warfarin (≦1 mg/day), and prophylactic low molecular weight heparin (LMWH) are permitted.
The subject has experienced any of the following within 3 months before the first dose of study treatment:
   clinically-significant hematemesis or lower gastrointestinal bleeding;
   hemoptysis of >0.5 (2.5 mL) teaspoon of red blood; or
   any other signs indicative of pulmonary hemorrhage.
The subject has radiographic evidence of cavitating pulmonary lesion(s) or tumor invading or encasing major blood vessels (Retroperitoneal and mediastinal lymphadenopathy abutting major blood vessels is not an exclusion criteria)
The subject has uncontrolled, significant intercurrent or recent illness including, but not limited to, the following conditions:
   Cardiovascular disorders including
   a) Congestive heart failure (CHF): New York Heart Association (NYHA) Class III (moderate) or Class IV (severe) at the time of screening
   b) Concurrent uncontrolled hypertension defined as sustained BP>140 mm Hg systolic, or >90 mm Hg diastolic despite optimal antihypertensive treatment (BP must be controlled at screening)
   c) Any of the following within 6 months before the first dose of study treatment:
      unstable angina pectoris
      clinically-significant cardiac arrhythmias
      stroke (including TIA, or other ischemic event)
      myocardial infarction
      thromboembolic event requiring therapeutic anticoagulation (Note: subjects with a venous filter (e.g. vena cava filter) are not eligible for this study)
   Gastrointestinal disorders (GI) particularly those associated with a high risk of perforation or fistula formation including:
      Any of the following at the time of screening
         intra-abdominal tumor/metastases invading GI mucosa
         active peptic ulcer disease,
         inflammatory bowel disease (including ulcerative colitis and Crohn's disease), diverticulitis, cholecystitis, symptomatic cholangitis or appendicitis
         Any of the following within 6 months before the first dose of study treatment:
         history of abdominal fistula
         gastrointestinal perforation
         bowel obstruction or gastric outlet obstruction
         intra-abdominal abscess. Note: Complete resolution of an intra-abdominal abscess must be confirmed prior to initiating treatment with cabozantinib even if the abscess occurred more than 6 months ago.
         GI surgery (particularly when associated with delayed or incomplete healing) within 28 days. Note: Complete healing following abdominal surgery must be confirmed prior to initiating treatment with cabozantinib even if surgery occurred more than 28 days ago.
   Other disorders associated with a high risk of fistula formation including PEG (polyethylene glycol) tube placement within 3 months before the first dose of study therapy or concurrent evidence of intraluminal tumor involving the trachea and esophagus.
Other clinically significant disorders such as:
   active infection requiring systemic treatment
   active or symptomatic viral hepatitis or chronic liver disease
   history of pituitary or adrenal dysfunction
   serious non-healing wound/ulcer/bone fracture
   history of organ transplant
   concurrent uncompensated hypothyroidism or thyroid dysfunction
   history of major surgery within 4 weeks or minor surgical procedures within 1 week before registration
   Requirement of steroids>5 mg per day
The subject is unable to swallow capsules or tablets
The subject has a corrected QT interval calculated by the Fridericia formula (QTcF)>500 ms within 28 days before randomization.
The subject has a previously identified allergy or hypersensitivity to components of the study treatment formulation.
The subject has had evidence within 2 years of the start of study treatment of another malignancy which required systemic treatment

### Inclusion of Women, Minorities and Other Underrepresented Populations

The only patient population which is excluded from this study is women by virtue of the fact that women do not get prostate cancer. All other populations are potentially eligible for enrollment.

### Treatment Plan

Treatment will be administered on an outpatient basis. Expected toxicities and potential risks as well as dose modifications for abiraterone and cabozantinib are described in Section 6 (Expected Toxicities and Dosing Delays/Dose Modifications). No investigational or commercial agents or therapies other than those described below may be administered with the intent to treat the participant's malignancy.

Patients will be required to maintain a medication diary. If patients miss a dose, and it is within 3 hours of the scheduled dose, they are to take the dose at that time. If it is after this window, they are to record the dose was missed and the reason why. If they vomit after taking the medication, they are not to make it up.

As summarized herein, patients will receive abiraterone at 1000 mg oral daily and will receive one of three possible doses of cabozantinib self-administered orally once daily per the dose level assignment in Part A or Part B. A cycle will be 28 days and will be repeated every 28 days in the absence of disease progression or unacceptable toxicity. Disease assessments will be monthly by history and physical and PSA measurements, and imaging will be every 8 weeks. Prednisone will be taken concurrently with abiraterone acetate at a dose of 5 mg twice daily with food. If prednisone is not available, prednisolone will be substituted. If abiraterone is held, prednisone dosing will be continued unless clinical management dictates otherwise.

The study will be conducted in 2 parts. Part A is phase 1 dose-escalation part to establish the MTD of cabozantinib in combination with abiraterone (1000 mg). This will be a standard 3+3 design.

| Dose Level | Cabozantinib Dose |
|---|---|
| 1 | 20 mg |
| 2 | 40 mg |
| 3 | 60 mg |

Part B will be conducted once safe and tolerable dose levels have been determined in Part A using the 3+3 design. As such, up to 3 dose levels may be expanded with a maximum of 12 subjects in each dose level. There will be no more than 12 patients accrued to a dose level, and this total includes the subjects from Part A. If the MTD is at the 20 mg dose level of cabozantinib with 1000 mg abiraterone, then only this dose level will be expanded to 12 patients.

### Assignment to a Dose Level

For the dose escalation portion, a patient will be assigned to a dose level according to progress of the trial and the 3+3 design. For a dose level, no more than 2 patients will be commenced in the same 7 day period.

For the expansion cohorts, patients will be sequentially assigned to a dose level starting with the cohort with the lowest dose level with an available slot. The purpose of this is to attempt to mitigate any possible bias that might be introduced by patient selection (e.g., only patients with high KPS for the higher dose level). The next patient to be treated will be assigned to the next available higher dose, and then once the highest dose level is studied, the sequential enrollment will re-start at the lowest available dose level.

For example, if after completion of part A there were 3 patients in each dose level, then the first patient registered in the expansion cohort will be enrolled to 20 mg cabozantinib dose level, the next patient to the 40 mg cabozantinib dose level, the third patient to the 60 mg cabozantinib dose level, and the fourth patient 20 mg cabozantinib dose level, and so forth. If during the course of the study, the 40 mg dose level has 12 evaluable patients accrued first (e.g., because it had 6 patients in part A), patients will be sequentially assigned to 20 mg and 60 mg cabozantinib dose level based upon registration until 12 evaluable patients are in all dose levels.

### Pre-Treatment Criteria

### Cycle 1, Day 1

For patients to commence protocol therapy, they must meet eligibility criteria. Baseline safety renal, liver, and CBC studies will need to be repeated if longer than 7 days prior to protocol therapy.

### Subsequent Cycles

Retreatment for Day 1 of subsequent cycles will be according to the table in section 6.

### Agent Administration

### Abiraterone Acetate

All patients enrolled will be instructed to take four 250 mg tablets (total of 1000 mg) orally (PO) of abiraterone daily. The patients will fast for 2 hours before their dose and continue to fast for 1 hour after their dose.

### LHRH Analogue Therapy or Surgical Castration

All patients will be required to demonstrate a castrate level of testosterone upon study entry. Whatever class of therapy (FDA approved analogue therapy) or surgical castration was used to induce the castrate state will be required for the duration of the study.

### Prednisone

Patients taking abiraterone acetate will be instructed to take 5 mg prednisone, twice daily with food. If prednisone is not available, prednisolone will be substituted.

### Cabozantinib

Cabozantinib will be supplied as 20 mg tablets. Subjects will receive cabozantinib orally administered daily at their assigned starting dose and will take it on an empty stomach at the same time as abiraterone (i.e., must fast for 2 hours before their dose and continue to fast for 1 hour after their dose).

### Restrictions

The concurrent administration of other anticancer therapy, including cytotoxic, hormonal (except LHRH agonists), or immunotherapy, is prohibited during study treatment. Use of other investigational drug therapy for any reason is prohibited. Concomitant therapy with any of the following listed is prohibited (or used with caution as indicated):
5 α-reductase inhibitor;
Chemotherapy;
Immunotherapy;
Ketoconazole, diethylstilbestrol, PC-SPES, and other preparations such as saw palmetto thought to have endocrine effects on prostate cancer;
Radiopharmaceuticals such as strontium (89Sr) or samarium (153Sm);
Aldactone, Spironol (spironolactone); and
Venlafaxine (can be used with caution for treatment of hot flashes).

The decision to administer a prohibited drug or treatment should be made based on the consideration of the safety of study participant. Patients who require the use of any of these agents may be discontinued from study-treatment.

### Anticoagulant Therapy

Patients who are taking warfarin may not participate in this trial. However, if an event happens on study, low molecular weight heparin is allowed. Therapeutic warfarin is not allowed, but low dose prophylactic warfarin is allowed.

### Anti-Emetics/Anti-Diarrheals

Prophylactic anti-emetics and/or anti-diarrheals will not routinely be given. Should a patient develop nausea, vomiting, and/or diarrhea, which, in the investigator's opinion, is considered related to the study medication, then appropriate prophylactic treatment may be given. The reason(s) for the use, doses, and dates of treatment should be recorded in the patient's medical records and appropriate section of the eCRF.

All medications (prescriptions or over-the-counter medications) continued at the start of the trial or started during the trial or until 30 days from the end of the last protocol treatment and different from the trial medication must be documented.

### Administration of Other Anti-Cancer Agents

Patients must not receive any other concurrent anti-cancer therapy, including investigational agents, while on study treatment.

### Other Medications

Other medications considered necessary for the patient's safety and well-being may be given at the discretion of the investigator and recorded in the appropriate sections of the Case Report Form.

### Duration of Therapy

Duration of therapy will depend on individual response, evidence of disease progression, and tolerance. In the absence of treatment delays due to adverse events, treatment may continue until one of the following criteria applies:

Disease progression is defined as follows:
PCWG (Prostate Cancer Working Group) defined radiographic or symptomatic progression (not prostate specific antigen, or PSA, progression)
   Progression events will be death, radiographic progression, and skeletal-related events. Radiographic progression is defined by RECIST 1.1 for soft tissue disease, or the appearance of two or more new bone lesions on bone scan. Progression at the first scheduled reassessment at Week 8 requires a confirmatory scan at least 6 weeks later unless it is accompanied by clear cut symptomatic disease progression. Skeletal-related events (defined as radiation therapy or surgery to bone, pathologic bone fracture, spinal cord compression, or change of antineoplastic therapy to treat bone pain) will also be recorded and if related to disease progression, detailed as such;
Unacceptable toxicity not managed with treatment, prophylaxis or dose modification;
Any symptomatic progression;
Intercurrent illness that prevents further administration of treatment;
Unacceptable adverse event(s) not managed with dose reductions;
Participant patient's request to discontinue study treatment (and allows follow-up);
Patient withdraws from study;
General or specific changes in the participant's condition render the participant unacceptable for further treatment in the opinion of the treating investigator;
Death from any cause.

### Drug Formulation and Administration

### Cabozantinib

Cabozantinib which will be supplied as 20 mg yellow film coated round tablets as summarized in the following table.

Cabozantinib Tablet Components and Composition

| Ingredient | Function | % w/w |
|---|---|---|
| Cabozantinib Drug Substance (25% drug load as | Active | 31.7 |
| free base) | Ingredient | |
| Microcrystalline Cellulose (Avicel PH-102) | Filler | 38.9 |
| Lactose Anhydrous (60M) | Filler | 19.4 |
| Hydroxypropyl Cellulose (EXF) | Binder | 3.0 |
| Croscarmellose Sodium (Ac-Di-Sol) | Disenegrant | 6.0 |
| Colloidal Silicon Dioxide, | Glidant | 0.3 |
| Magnesium Stearate | Lubricant | 0.75 |
| Opadry Yellow Film Coating which includes: | | |
| HPMC 2910/Hypromellose 6 cp | | |
| Titanium dioxide | Film Coating | 4.00 |
| Triacetin | | |
| Iron Oxide Yellow | | |

Cabozantinib is administered once daily as an oral tablets(s). Subjects will be provided with a sufficient supply of study treatment and instructions for taking the study treatment on days without scheduled clinic visits. After fasting (with exception of water) for 2 hours, subjects will take study treatment daily each morning with a full glass of water (minimum of 8 oz/240 mL) and continue to fast for 1 hour after each dose of study treatment. If doses are withheld, the original schedule of assessments should be maintained when cabozantinib is restarted.

If patients miss a dose, and it is within 3 hours of the scheduled dose, they are to take the dose at that time. If it is after this window, they are to record the dose was missed and the reason why. If they vomit after taking the medication, they are not to make it up.

Each patient will be provided with a 30-day supply to allow for visits to occur every 28 days with a ±2 day window.

### Abiraterone

Abiraterone acetate 250 mg tablets are oval, white to off-white and contain abiraterone acetate and compendial (USP/NF/EP) grade lactose monohydrate, microcrystalline cellulose, croscarmellose sodium, povidone, sodium lauryl sulfate, magnesium stearate, colloidal silicon dioxide, and purified water, in descending order of concentration (the water is removed during tabletting).

Abiraterone is administered once daily as oral tablet(s). Subjects will be provided with a sufficient supply of study treatment and instructions for taking the study treatment on days without scheduled clinic visits. After fasting (with exception of water) for 2 hours, subjects will take study treatment daily each morning with a full glass of water (minimum of 8 oz/240 mL) and continue to fast for 1 hour after each dose of study treatment. If doses are withheld, the original schedule of assessments should be maintained when abiraterone is restarted.

If patients miss a dose, and it is within 3 hours of the scheduled dose, they are to take the dose at that time. If it is after this window, they are to record the dose was missed and the reason why. If they vomit after taking the medication, they are not to make it up.

### Correlative/Special Studies

### Pharmacokinetic Studies

Serum will be collected as indicated in the study calendar.

### Cabozantinib Levels

Fill 4 mL K3 EDTA (ethylenediaminetetraacetic acid) lavender top tube completely, and mix immediately by gently inverting at least 8 to 10 times. Record the exact time (24-hour clock) of sample collection. Centrifuge in 4° C. at a minimum of 1200×6 for 15 minutes until cells and plasma are separated. Use pipettes to transfer plasma into 3 mL polypropylene tubes. Freeze and store at −70° C. When shipping, ship on dry ice.

### Abiraterone Levels

Plasma will be drawn at the timepoints indicated in the study calendar. Sample processing must be started within 30 minutes of blood draw. Draw approximately 10 mL of blood into the two provided lavender top vacutainer tubes. Invert gently, and then centrifuge them at 3500 rpm for 30 minutes. Without delay, divide plasma equally into six (6) labeled cryovials in ˜1 ml aliquots, and then freeze the samples immediately at −70° C. If a −70° C. freezer is not available, plasma samples may be stored at −20° C. until shipped. Samples MUST be shipped as soon as possible and sent by overnight courier. Do not allow samples to thaw.

### Pharmacodynamic Studies

### Imaging Procedural Information

All patients enrolled in the study will be evaluated with 99mTc-MDP skeletal scintigraphy, whole body 18F-FDG PET/CT, 18F-NaF PET/CT, and contrast-enhanced diagnostic CT of the abdomen and pelvis according to the schedule shown in the study calendar. It is anticipated that the PET studies, bone scans, and diagnostic CT studies will be obtained within a few days of each other, preferably over a 2-3 day period. Tumor assessments should be done at baseline and 8 weeks following the first dose of study treatment. The bone scans will be centrally reviewed by an imaging CRO (MedQIA). All other imaging studies will be centrally reviewed by the DFCI core laboratory. The accompanying Radiology Study Procedural Guidelines details the imaging protocols for each modality. In essence the ⁹⁹mTc-MDP skeletal scintigraphy will be performed first and if disease is outside of the planned ¹⁸F-NaF PET/CT usual imaging (i.e., above skull base or below thighs), the 18F-NaF PET/CT imaged areas will be expanded.

### Details of Peripheral Blood Collection

### CTC Collection

CTC enumeration will be performed at a central laboratory using the analytically valid. CellSearch system (Veridex, LLC). For patients with baseline CTC counts of ≧5 cells/7.5 mL of blood, a conversion is defined as a decline in the CTC count to <5 cells/7.5 mL of blood. CTCs will be collected according to standard protocol using the collection kit provided.

For molecular characterization of the CTCs, at the appropriate time three samples of 3 mL will be taken from patients at the indicated time points. The cells will be filtered using the Screen Cell filtration device and stored frozen until ready for processing by the pathology core. Following incubation at the appropriate temperature with a lysis buffer, the Eppendorf tube containing the capsule-filter is centrifuged for 1 min at 12,000×g and the capsule-filter removed and discarded. The flow-through is stored in the closed Eppendorf tube and stored until ready for analysis. The DFCI Core facility (Translational Research Facility) has used this assay in non-small cell lung cancer and been also able to detect EGFR mutations and stored frozen until ready for processing by the pathology core. The three samples will allow for quality control to characterize the circulating cells as prostate cancer by IHC (immunohistochemical) staining for PSA and CD45. If the CTC is PSA positive and CD45 negative, we will co-stain for proteins relevant to the abiraterone (CYP17A1) and cabozantinib (phospho-cMET). If the cells are PSA negative (which can occur with prostate cancer), we will only call these cells prostate cancer if PSMA (+), cytokeratin (+), and CD45 (−).

### DEFINITIONS

Evaluable for toxicity. All participants who receive at least one dose of study treatment will be evaluable for toxicity from the time of their first treatment.

Evaluable for objective response. Only those participants who have measurable disease present at baseline, have received at least one cycle of therapy, and have had their disease re-evaluated will be considered evaluable for response. These participants will have their response classified according to the definitions stated below. (Note: Participants who exhibit objective disease progression or die prior to the end of cycle 1 will also be considered evaluable).

### Disease Parameters

Measurable Disease.

Measurable lesions are defined as those that can be accurately measured in at least one dimension (longest diameter to be recorded) as ≧20 mm by chest x-ray, as ≧10 mm with CT scan, or ≧10 mm with calipers by clinical exam. All tumor measurements must be recorded in millimeters (or decimal fractions of centimeters). Tumor lesions that are situated in a previously irradiated area might be considered measurable if shown to progress since the radiation.

Malignant Lymph Nodes.

To be considered pathologically enlarged and measurable, a lymph node must be ≧15 mm in short axis when assessed by CT scan (CT scan slice thickness recommended to be no greater than 5 mm). At baseline and in follow-up, only the short axis will be measured and followed.

Non-Measurable Disease.

All other lesions (or sites of disease), including small lesions (longest diameter<10 mm or pathological lymph nodes with ≧10 to <15 mm short axis, are considered non-measurable disease. Bone lesions, leptomeningeal disease, ascites, pleural/pericardial effusions, lymphangitis cutis/pulmonis, inflammatory breast disease, abdominal masses identified by physical exam that are not measurable by reproducible imaging techniques, and cystic lesions are all considered non-measurable. Cystic lesions that meet the criteria for radiographically defined simple cysts should not be considered as malignant lesions (neither measurable nor non-measurable) since they are, by definition, simple cysts. ‘Cystic lesions’ thought to represent cystic metastases can be considered as measurable lesions if they meet the definition of measurability described above. However, if non-cystic lesions are present in the same patient, these are preferred for selection as target lesions.

Target Lesions.

All measurable lesions up to a maximum of 2 lesions per organ and 5 lesions in total, representative of all involved organs, should be identified as target lesions and recorded and measured at baseline. Lesions must be accurately measured in 1 dimension with a minimum size of 10 mm by CT or MRI (slice thickness no greater than 5 mm), 20 mm by chest x-ray. Nodes must have a short axis≧15 mm. The short axis should be included in the sum of the lesions in the calculation of response. Nodes that shrink to <10 mm are considered normal. Target lesions should be selected on the basis of their size, be representative of all the involved organs, and should be lesions that can be followed with reproducible repeated measurements.

Lytic bone lesions or mixed lytic-blastic lesions, with identifiable soft tissue components, that can be evaluated by cross sectional imaging techniques such as CT or MRI can be considered target lesions if the soft tissue component meets the definition of measurability as defined above. Cystic lesions thought to represent cystic metastases can be considered as target lesions. However, if non-cystic lesions are present, these are preferred for selection as target lesions. Lesions in previously irradiated areas or areas subject to other loco-regional therapy are usually not considered measurable unless there has been demonstrated progression of that lesion.

Non-Target Lesions.

All other lesions, including small lesions<10 mm or pathological lymph nodes measuring>10 mm to <15 mm in short axis, as well as truly non-measurable lesions, which include leptomeningeal disease, ascites, pleural or pericardial effusion, inflammatory breast disease, lymphangitic involvement of skin or lung, or abdominal masses identified by physical exam that are not measurable by reproducible imaging techniques.

### Methods for Evaluation of Measurable Disease

All measurements should be taken and recorded in metric notation using a ruler or calipers. All baseline evaluations should be performed as closely as possible to the beginning of treatment and never more than 4 weeks before the beginning of the treatment.

The same method of assessment and the same technique should be used to characterize each identified and reported lesion at baseline and during follow-up. Imaging-based evaluation is preferred to evaluation by clinical examination unless the lesion(s) being followed cannot be imaged but are assessable by clinical exam.

Clinical Lesions.

Clinical lesions will only be considered measurable when they are superficial (e.g., skin nodules and palpable lymph nodes) and mm diameter as assessed using calipers (e.g., skin nodules). In the case of skin lesions, documentation by color photography, including a ruler to estimate the size of the lesion, is recommended.

Chest x-Ray.

Lesions on chest x-ray are acceptable as measurable lesions when they are clearly defined and surrounded by aerated lung. However, CT is preferable.

Conventional CT and MRI.

This guideline has defined measurability of lesions on CT scan based on the assumption that CT slice thickness is 5 mm or less. If CT scans have slice thickness greater than 5 mm, the minimum size for a measurable lesion should be twice the slice thickness. MRI is also acceptable in certain situations (e.g., for body scans).

PET-CT.

At present, the low dose or attenuation correction CT portion of a combined PET-CT is not always of optimal diagnostic CT quality for use with RECIST measurements. However, if the site can document that the CT performed as part of a PET-CT is of identical diagnostic quality to a diagnostic CT (with IV and oral contrast), then the CT portion of the PET-CT can be used for RECIST measurements and can be used interchangeably with conventional CT in accurately measuring cancer lesions over time. Note, however, that the PET portion of the CT introduces additional data which may bias an investigator if it is not routinely or serially performed.

FDG-PET.

While FDG-PET response assessments need additional study, it is sometimes reasonable to incorporate the use of FDG-PET scanning to complement CT scanning in assessment of progression (particularly possible ‘new’ disease). New lesions on the basis of FDG-PET imaging can be identified according to the following algorithm:
a. Negative FDG-PET at baseline, with a positive FDG-PET at follow-up, is a sign of PD based on a new lesion.
b. No FDG-PET at baseline and a positive FDG-PET at follow-up: If the positive FDG-PET at follow-up corresponds to a new site of disease confirmed by CT, this is PD. If the positive FDG-PET at follow-up is not confirmed as a new site of disease on CT, additional follow-up CT scans are needed to determine if there is truly progression occurring at that site (if so, the date of PD will be the date of the initial abnormal FDG-PET scan). If the positive FDG-PET at follow-up corresponds to a pre-existing site of disease on CT that is not progressing on the basis of the anatomic images, this is not PD.
c. FDG-PET may be used to upgrade a response to a CR in a manner similar to a biopsy in cases where a residual radiographic abnormality is thought to represent fibrosis or scarring. The use of FDG-PET in this circumstance should be prospectively described in the protocol and supported by disease-specific medical literature for the indication. However, it must be acknowledged that both approaches may lead to false positive CR due to limitations of FDG-PET and biopsy resolution/sensitivity.
   A ‘positive’ FDG-PET scan lesion means one which is FDG avid with an uptake greater than twice that of the surrounding tissue on the attenuation corrected image.

### Response Criteria

Complete Response (CR).

Disappearance of all target lesions. Any pathological lymph nodes (whether target or non-target) must have reduction in short axis to <10 mm.

Partial Response (PR).

At least a 30% decrease in the sum of the diameters of target lesions, taking as reference the baseline sum diameters.

Progressive Disease (PD).

At least a 20% increase in the sum of the diameters of target lesions, taking as reference the smallest sum on study (this includes the baseline sum if that is the smallest on study). In addition to the relative increase of 20%, the sum must also demonstrate an absolute increase of at least 5 mm. (Note: the appearance of one or more new lesions is also considered progressions).

Stable Disease (SD).

Neither sufficient shrinkage to qualify for PR nor sufficient increase to qualify for PD, taking as reference the smallest sum diameters while on study

Definition of New Lesion.

The finding of a new lesion should be unequivocal (i.e., not due to difference in scanning technique, imaging modality, or findings thought to represent something other than tumor (ex: new bone lesions may be healing or flare of pre-existing lesions). However, a lesion identified on a follow-up scan in an anatomical location that was not scanned at baseline is considered new and will indicate PD. If a new lesion is equivocal (because of small size, etc.), follow-up evaluation will clarify if it truly represents new disease, and if PD is confirmed, progression should be declared using the date of the initial scan on which the lesion was discovered.

### Evaluation of Non-Target Lesions

Complete Response (CR).

Disappearance of all non-target lesions and normalization of tumor marker level. All lymph nodes must be non-pathological in size (<10 mm short axis). If tumor markers are initially above the upper normal limit, they must normalize for a patient to be considered in complete clinical response.

Non-CR/Non-PD.

Persistence of one or more non-target lesion(s) and/or maintenance of tumor marker level above the normal limits.

Progressive Disease (PD).

Appearance of one or more new lesions (new lesions must be >slice thickness) and/or unequivocal progression of existing non-target lesions. Overall level of substantial worsening that merits discontinuation of therapy. A useful test that can be applied when assessing non-targets for unequivocal progression is to consider if the increase in overall disease burden based on the change in non-measurable disease is comparable in magnitude to the increase that would be required to declare PD for measurable disease.

Unknown (UN).

Assessment of non-target lesions cannot be made due to insufficient or unevaluable data. In this case, a concise explanation must be given.

Definition of New Lesion.

The finding of a new lesion should be unequivocal (i.e., not due to difference in scanning technique, imaging modality, or findings thought to represent something other than tumor, e.g., new bone lesions may be healing or flare of pre-existing lesions). However, a lesion identified on a follow-up scan in an anatomical location that was not scanned at baseline is considered new and will indicate PD. If a new lesion is equivocal (because of small size, etc.), follow-up evaluation will clarify if it truly represents new disease and if PD is confirmed, progression should be declared using the date of the initial scan on which the lesion was discovered.

### Evaluation of Best Overall Response

The best overall response is the best response recorded from the start of the treatment until disease progression/recurrence (taking as reference for progressive disease the smallest measurements recorded since the treatment started). The patient's best response assignment will depend on the achievement of both measurement and confirmation criteria.

For Patients with Measurable Disease (i.e., Target Disease):

| | | | | Best Overall Response |
|---|---|---|---|---|
| Target | Non-Target | New | Overall | when Confirmation is |
| Lesions | Lesions | Lesions | Response | Required* |
| CR | CR | No | CR | ≧4 wks. |
| | | | | Confirmation** |
| CR | Non-CR/Non-PD | No | PR | ≧4 wks. |
| | | | | Confirmation** |
| CR | Not evaluated | No | PR | |
| PR | Non-CR/Non- | No | PR | |
| | PD/not evaluated | | | |
| SD | Non-CR/Non- | No | SD | documented at least |
| | PD/not evaluated | | | once ≧4 wks. from |
| | | | | baseline** |
| PD | Any | Yes or | PD | no prior SD, PR or CR |
| | | No | | |
| Any | PD*** | Yes or | PD | |
| | | No | | |
| Any | Any | Yes | PD | |

*See RECIST 1.1 manuscript for further details on what is evidence of a new lesion.
**Only for non-randomized trials with response as primary endpoint.
***In exceptional circumstances, unequivocal progression in non-target lesions may be accepted as disease progression.

For Patients with Non-Measurable Disease (i.e., Non-Target Disease):

| Non-Target Lesions | New Lesions | Overall Response |
|---|---|---|
| CR | No | CR |
| Non-CR/non-PD | No | Non-CR/non-PD* |
| Not all evaluated | No | not evaluated |
| Unequivocal PD | Yes or No | PD |
| Any | Yes | PD |

*‘Non-CR/non-PD’ is preferred over ‘stable disease’ for non-target disease since SD is increasingly used as an endpoint for assessment of efficacy in some trials so to assign this category when no lesions can be measured is not advised

### Duration of Response

Duration of Overall Response.

The duration of overall response is measured from the time measurement criteria are met for CR or PR (whichever is first recorded) until the first date that recurrence or PD is objectively documented, taking as reference for PD the smallest measurements recorded since the treatment started.

Duration of Overall Complete Response.

The duration of overall CR is measured from the time measurement criteria are first met for CR until the first date that recurrent disease is objectively documented.

Duration of Stable Disease:

Stable disease is measured from the start of the treatment until the criteria for progression are met, taking as reference the smallest measurements recorded since the treatment started.

### Other Response Parameters

Progression-free survival is defined as time from registration to the earliest objective evidence of progression (either radiographic or skeletal-related event) or death due to any cause. Patients will be assessed for objective disease progression at regularly scheduled visits. The consensus guidelines of the Prostate Cancer Clinical Trials Working Group 2 have been taken into consideration for the determination of disease progression.

Radiographic disease progression is defined by RECIST 1.1 for soft tissue disease or the appearance of two or more new bone lesions on bone scan. Progression in the absence of clear symptomatic worsening at the first scheduled reassessment at Week 8 requires a confirmatory scan 6 or more weeks later. Only the standard imaging procedures, technetium bone scans and CT scans with reports from the TIMC, will be used for measurement of effect. This study also investigates the use of NaF and FDG-PET to measure radiographic effect.

The time to first skeletal-related event is defined as time from registration to the occurrence of the first skeletal-related event. Patients will be assessed for skeletal-related events at regularly scheduled visits. A skeletal-related event is defined as radiation therapy or surgery to bone, pathologic bone fracture, spinal cord compression, or change of antineoplastic therapy to treat bone pain.

Patients who do not reach the endpoint will be right censored at their last assessment.

PSA progression will be assessed for each patient in the study as defined above. Time to PSA progression is defined as time from randomization to PSA progression. Patients who do not reach the endpoint will be right censored at their last assessment.

CTC conversion will be assessed for patients with baseline CTC counts of ≧5 cells/7.5 mL of blood. A conversion is defined as a decline in the CTC count to <5 cells/7.5 mL of blood. Conversion rates will be assessed for all patients and across dose levels.

### Statistical Analysis

The number and percent of patients experiencing a DLT, with two-stage Exact Binomial Confidence Interval (CI) for the DLT rate of each dose level will be obtained. Toxicities will be summarized as number and percent of patients, overall and according to maximum grade experienced, separately for each dose level. Distributions of time to progression, treatment failure and overall survival will be estimated using Kaplan-Meier method. Other antitumor measures will be summarized descriptively.

Changes in CTC number will be reported according to cabozantinib dose level descriptively as number of patients converted from ≧5 to <5 and for the total population. For each dose level, the percent decline in markers of bone turnover and microenvironment will be reported descriptively with 90% exact CI. Conservatively assuming that the standard deviation of percent change is 40%, the estimated width of 90% CI on a percent decline of PD markers from baseline for evaluating 12 patients is +/−15%. Progression free and overall survival will be assessed using Kaplan Meier plots.

For radiological studies, a comparison between all three scans will be undertaken considering all patients together. The DFCI TIMC will use RECIST criteria to assess changes using CT imaging and will also assess determine regions of interest (ROI) and changes from SUVmax from the FDG-PET and NaF PET. For the technetium bone scans, data will be generated by MedQIA for semi-quantitative analysis of bone scan changes. For each PET modality, efficacy measures will be assessed for sensitivity, specificity, accuracy and predictive values and correlated with time to progression and overall survival using the definitions in Section 10 and detailed in Appendix III.

### Imaging Procedural Information

All patients enrolled in the study will be evaluated with ^{99m}Tc-MDP skeletal scintigraphy, whole body ¹⁸F-FDG PET/CT, ¹⁸F-NaF PET/CT, and contrast-enhanced diagnostic CT of the abdomen and pelvis according to the schedule shown in the study calendar. It is anticipated that the PET studies, bone scans and diagnostic CT studies will be obtained within a few days of each other, preferably over a 2-3 day period. Tumor assessments should be done at baseline and 8 weeks following the first dose of study treatment. The ^{99m}Tc-MDPbone scans will be centrally reviewed by an imaging CRO (MedQIA). All other imaging studies will be centrally reviewed by the DFCI core laboratory.

### Image Acquisition Parameters

Imaging acquisition parameters should follow the local standard of care when possible, within the constraints detailed below.

Whole body bone scans should be acquired in the anterior and posterior projections approximately 3 hours following the IV administration of approximately 20 mCi^{99m}Tc-methylene diphosphonate (MDP). The imaging parameters should be consistent between baseline and follow-up time points. In specific cases of symptoms suggesting cord compression or impending fracture, additional imaging may be warranted such as radiographs or MRI. All ^{99m}Tc-MDPbone scan images should be submitted to MedQIA for review.

¹⁸F-FDG PET/CT scan acquisition should follow the NCI Guidelines. Patients should avoid strenuous exercise for 24 hours prior to the study and should fast for 4-6 hours prior to the study depending on their diabetic status. A serum glucose less than 200 mg/dL at the time of FDG injection is recommended. A typical adult patient should receive approximately 14mCi FDG IV followed by an uptake period of 60 minutes. It is recommended that for a typical adult patient, each scan be acquired from the skull vertex to the pelvis, unless otherwise indicated, using approximately 7 bed positions with 4 minute acquisitions per bed position. Patient preparation and imaging parameters must remain consistent throughout all scans while on protocol. PET images should be reconstructed with an iterative approach (e.g., OSEM, RAMLA). The CT attenuation scan should also be reconstructed and all reconstructed images should be submitted to the DFCI core laboratory for review.

¹⁸F-NaF PET/CT scans should be obtained 30-60 minutes after the IV injection of approximately 10 mCiNaF. There is no specific patient preparation required prior to the study; however, patients should be encouraged to drink 500-1000 ml of plain water shortly prior to the study and up to 500 ml of water shortly after the radiopharmaceutical administration. It is recommended that images be obtained from the skull vertex to pelvis, unless otherwise indicated, using approximately 8 bed positions with 4 minute acquisitions per bed position. Patient preparation and imaging parameters must remain consistent throughout all scans while on protocol. The PET should be reconstructions using filtered back projection nor an iterative approach (i.e. OSEM). The CT attenuation scan should also be reconstructed and all reconstructed images should be submitted to the DFCI core laboratory for review.

Diagnostic CT scans should include full coverage of the abdomen and pelvis and be obtained following IV contrast administration. A pre-contrast scan is not required. CT acquisition collimation should be less than or equal to 3 mm, with reconstructed axial images provided at 5 mm or less slice thickness in a soft tissue kernel. The reconstructed series should be submitted to the DFCI core laboratory. The same technique should be used at baseline and follow-up. Please note, the diagnostic CT study is in addition to the non-diagnostic attenuation correction CT performed as part of the PET study.

### Bone Scan Assessment by MedQIA

Of note, the bone scans will be categorized into one of three categories:
1. Normal/benign: physiologic uptake or uptake typical for a benign process such as degenerative joint disease.
2. Positive for osseous metastases: uptake typical for metastatic disease.
3. Equivocal: uptake could not be confidently categorized as 1 or 2 above.

For each positive bone scan, the positive area on the bone scan (PABS) will be computed using semi-automated CAD software which segments each lesion based on image intensity and then sums the lesion(s) to provide an overall measure of tumor burden. Response will be determined based on percent change from baseline in the Bone Scan Area (% BSA) as detailed in the MedQIA Charter.

### Assessment of FDG-PET, NaF-PET by DFCI Core Lab and CT Scans by TIMC

Quantification of the change in FDG tumor uptake and in NaF skeletal lesion uptake may provide an early, sensitive, pharmacodynamic marker of the tumoricidal effect of cabozantinib. For each study, index lesions will be identified based on the highest metabolic activity, analyzed and quantified using SUVmax at baseline and follow-up (maximum 10 lesions). Metabolic response will be classified using EORTC criteria based on thresholds for % SUVmax change relative to baseline and using a mean SUV based on a 70% threshold of the maximum SUV. This will be performed by the DFCI core lab. Anatomic tumor response will be classified according to the best response achieved using RECIST applied to the diagnostic CT and performed by TIMC.

### Comparisons of Bone Scan, NaF-PET, and FDG-PET Scans

It is estimated that the study accrue up to 35 patients, and that a large fraction of this number will have all 3 scans, including whole body FDG-PET/CT, whole body NaF PET and Bone scan suitable for analysis.

### Statistical Considerations:

Objective 1:

To determine a semiquantitative value in bone scintigraphy uptake from baseline to the week 8 scan which correlates with progression-free survival (PFS).

PFS is defined as time from registration to documented first PSA progression, radiographic or symptomatic progress or to death without progression. Patients without documented progression or death reported will be censored at the time of the last documented disease evaluation. Assuming that hazards for progression are proportional over the unit increment of the percent change in bone scintigraphy measurement (% BSA), we will estimate the coefficient of % BSA using a Cox proportional hazards model. Approximately, a Cox regression of the log hazard ratio on the percent change (standard deviation 40%) with 35 patients achieves 92% power to detect a regression coefficient equal to 0.02 (hazard ratio 1.02 per unit increment of % BSA) at a significance level of 0.05. The sample size was adjusted for an anticipated event rate of 0.50 and a two-sided test was used. If we accrue at least 24 patients, there is 79% power for the same statistical test. Based on the estimated coefficient, we will find cutoff percent change value(s) that corresponds to an optimal PFS median difference.

The study also compute sensitivity and specificity with 95% confidence intervals for standard clinical parameters including PSA, CT and standard bone scintigraphy report using formulae by Altman and Bland. All evaluation grades will be binary re-categorized for computing sensitivity and specificity in an exploratory fashion.

Objective 2:

To determine a percent change in SUVmax of on FDG PET from baseline to the week 8 scan (percent change in sum of SUVmax) correlates with PFS.

The study will convert percent change in sum of SUVmax to binary metabolic response based on EORTC criteria (CR+PR+SD vs. PD). Kaplan-Meier estimates will be used for event-time distributions, and PFS will be compared between CR+PR+SD vs. PD using log-rank tests. A one-sided log-rank test with a total sample size of 35 subjects yields approximately 91% power at a 0.05% significance level to detect a median PFS difference, 4 months in PD vs. 14 months in CR+PR+SD (hazard ratio 0.29), assuming that the study lasts for 24 months where subject accrual occurs in the first 18 months. If we accrue at least 24 patients, there is 81% power for the same statistical test.

The study will also compute sensitivity and specificity with 95% confidence intervals for metabolic response against standard clinical parameters including PSA, CT and standard bone scintigraphy report. All evaluation grades will be binary re-categorized for computing sensitivity and specificity.

Objective 3:

To determine a percent change in SUVmax of on NaF PET from baseline to the week 8 scan (percent change in sum of SUVmax) correlates with PFS.

The study will apply the statistical procedure described in Objective 2 to study the percent change in sum of SUVmax from baseline to the 8 week visit on PFS.

Objective 4:

To compare predictive value of bone scintigraphy versus FDG-PET versus Sodium Fluoride PET with regards to PFS.

Using the pre-chosen cutoffs in Objective 1, we will categorize % BSA into categorical response outcome and obtain Kaplan-Meier estimates stratified by the created outcome. The three methods (bone scintigraphy, FDG-PET, and Sodium Fluoride PET) will be compared using the results of log-rank tests based on the Kaplan-Meier method.

ROC curve area will be used for comparing the sensitivity and specificity of bone scintigraphy, FDG-PET, and Sodium Fluoride PET where the reference standard is positive/negative result of standard care CT scan.

In all evaluations, results with p-values<0.05 will be considered statistically significant.

### Clinical Factors Affecting Tumor Assessment

Clinical information that may influence the interpretation of imaging studies will be documented at baseline and at 8 weeks. Information to be collected should include:
Radiotherapy prior to or during study treatment;
Fracture/trauma prior to or during study treatment;
Infection prior to or during study treatment; and
Local intervention prior to or during study treatment, e.g., Resection and/or biopsy.

### Results

The results of the study are shown below. There were no DLTs in first 4 weeks of any of the dose levels, but 2 of 3 patients at the 60 mg cabozantinib dose level required dose reduction beyond cycle 2 due to fatigue. Table 1 shows the results for patients in the 20 mg cabozantinib cohort. Table 2 shows the results for patients in the 40 mg cabozantinib cohort. Table 3 shows the results for patients in the 60 mg cabozantinib cohort.

TABLE 1
Results for Patients at 20 mg Cabozantinib Dosage.

| Abiraterone 1 gm | Patient 1 | Patient 2 | Patient 3 |
|---|---|---|---|
| Cabozantinib 20 mg | Post-docetaxel | Post-docetaxel | Post-docetaxel |
| PSA BL | 405 | 1.35 | 10.5 |
| PSA Month-1 | 130 | 0.05 | 1.8 |
| PSA Month-2 | 98 | 0.01 | 0.59 |
| Lowest PSA | 49 on C5D1 | 0.01 @ C7D1 | 0.04 @ C7D1 |
| Last PSA | 108 @ C11D1 | 0.05 @ C9D1 | 0.05 @ C10D1 |
| CTC BL | 16 | 0 | 1 |
| CTC Month 2 | 1 | 0 | 0 |
| Notable AEs | None | None | None |
| Current Pt status | PD after 10 | Off cabozantinib | PSA and scans |
| | cycles on scans | with Non-cancer | stable at C10 |
| | | SRE at C9 | |

TABLE 2
Results for Patients at 40 mg Cabozantinib Dosage.

| | | | Patient 3 |
|---|---|---|---|
| Abiraterone 1 gm | Patient 1 | Patient 2 | Post-adjuv |
| Cabozantinib 40 mg | Post-docetaxel | Post-docetaxel | docetaxel |
| PSA BL | 125 | 26 | 0.07 |
| PSA Month-1 | 157 | 31 | 0.05 |
| PSA Month-2 | 284 | 48 | 0.07 |
| Lowest PSA | 125 @ C1D1 | 26 @ C1D1 | 0.05 @ C2D1 |
| Last PSA | 1041 @ 30 day | 52 @ C5D1 | 0.07 @ C4D1 |
| | f/u visit | | |
| CTC BL | 6 | 0 | 2 |
| CTC Month 2 | 10 | 0 | 0 |
| Notable AEs | BP elevation at | None | Intolerable G2 |
| | Day 29 - | | fatigue - Decr to |
| | cabozantinib | | 20 mg at C3 |
| | reduce to | | |
| | 20 mg at | | |
| | C2, D1 | | |
| Current Pt status | Progression at | Scans stable at | Scans stable at |
| | Day 56 | C4 | C4 |

TABLE 3
Results for Patients at 60 mg Cabozantinib Dosage.

| Abiraterone | | | |
|---|---|---|---|
| 1000 mg | | Patient 2 | Patient 3 |
| Cabozantinib | Patient 1 | No prior | No prior |
| 60 mg | Post-docetaxel | docetaxel | docetaxel |
| PSA BL | 10 | 8 | 3.69 |
| | (++liver, bone | | |
| | mets) | | |
| PSA Month-1 | 2.4 | 2.4 | 1.17 |
| PSA Month-2 | 4.8 | 1.6 | Too early |
| Lowest PSA | 2.4 | 1.6 | |
| Last PSA | | | |
| CTC BL | 44 | 0 | TBD |
| CTC Month 2 | 7 | Testing failed | TBD |
| Notable AEs | Muscle pain | Muscle pain | Tolerated cycle 1 |
| | Fatigue | Fatigue | well |
| | Pain in hand and | Pain in hand and | |
| | feet | feet | |
| | DVT | | |
| Dose tolerated | 40 mg | 40 mg | 60 mg |
| beyond cycle 1 | cabozantinib with | cabozantinib with | cabozantinib with |
| | 1000 mg | 1000 mg | 1000 mg |
| | abiraterone in | abiraterone at | abiraterone in |
| | cycle 2 | cycle 3 | cycle 2 |

All patients enrolled in the study were evaluated with ^{99m}Tc-MDP skeletal scintigraphy, whole body ¹⁸F-FDG PET/CT, ¹⁸F-NaF PET/CT, and contrast-enhanced diagnostic CT of the abdomen and pelvis according to the schedule shown in the study calendar. The PET studies, bone scans, and diagnostic CT studies were obtained within a few days of each other, preferably over a 2-3 day period. Tumor assessments were done at baseline and 8 weeks following the first dose of study treatment.

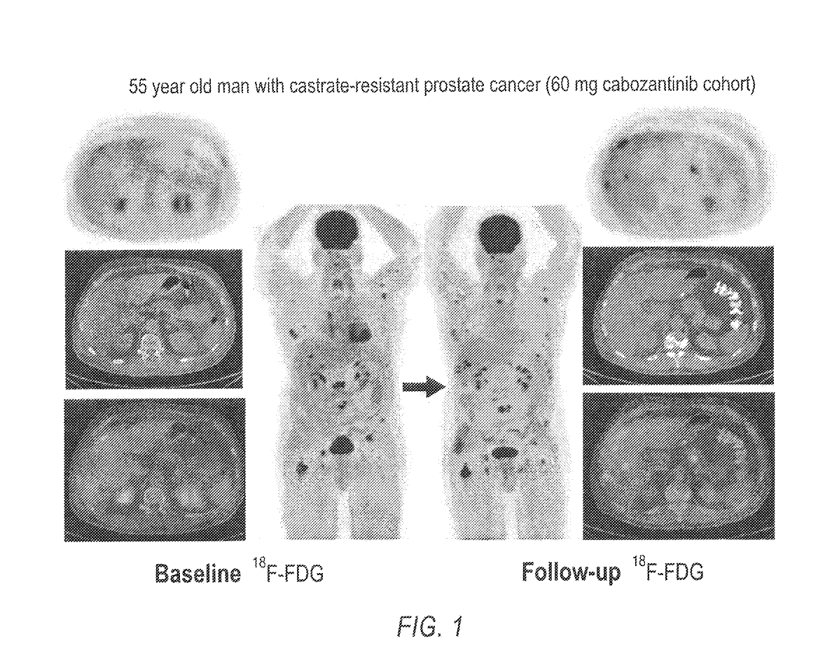

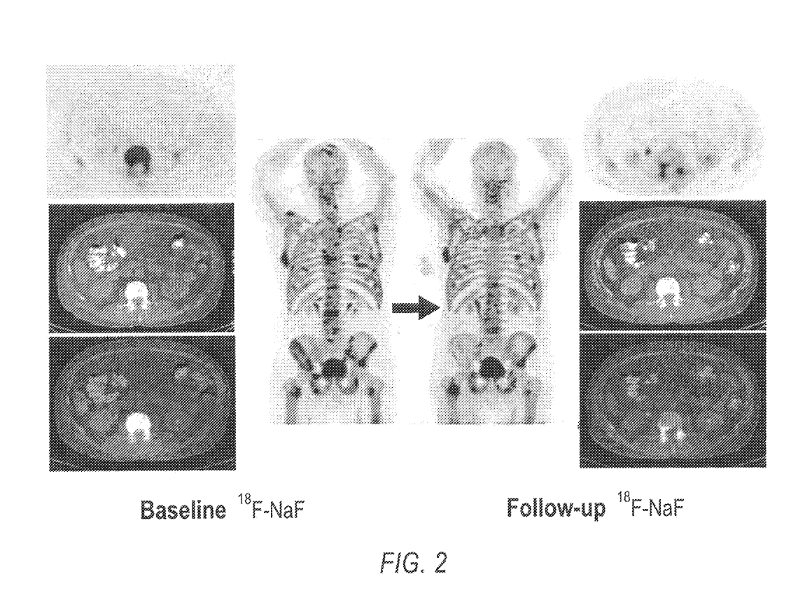

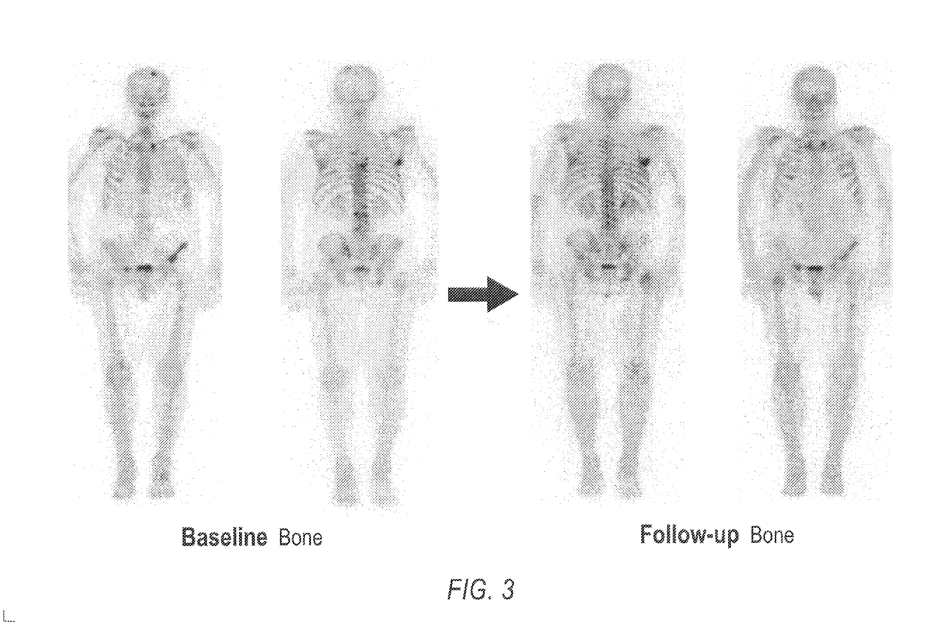

FIG. 1 shows the whole body ¹⁸F-FDG PET/CT scans at baseline and 8 weeks following the first dose of study treatment for a 55-year old man with castrate-resistant prostate cancer. This patient was from the 60 mg cabozantinib cohort. FIG. 2 shows the whole body ¹⁸F-NaF PET/CT scans for the same patient. FIG. 3 shows the whole body bone scans for the same patient.

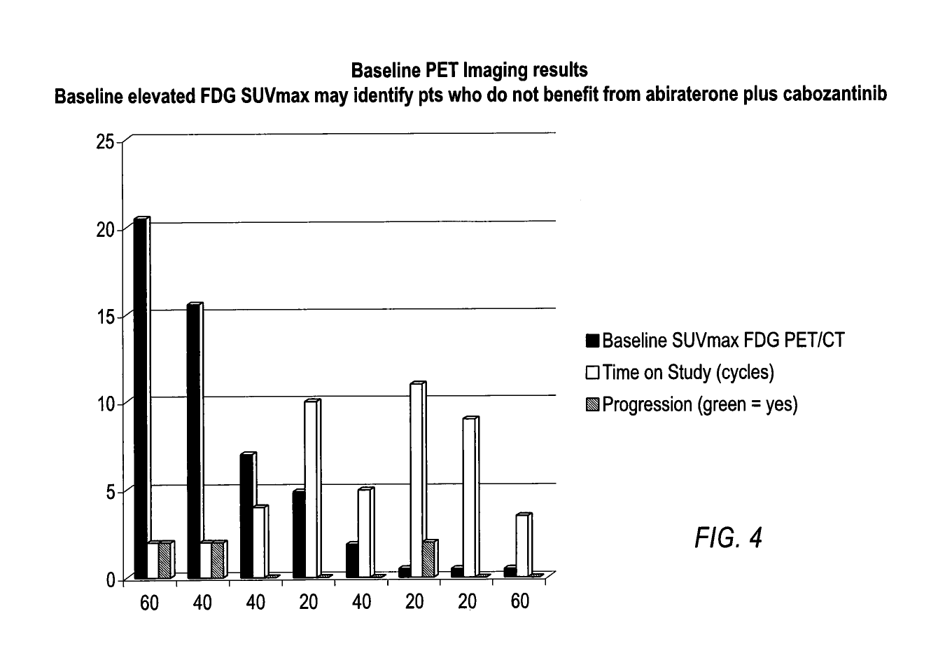

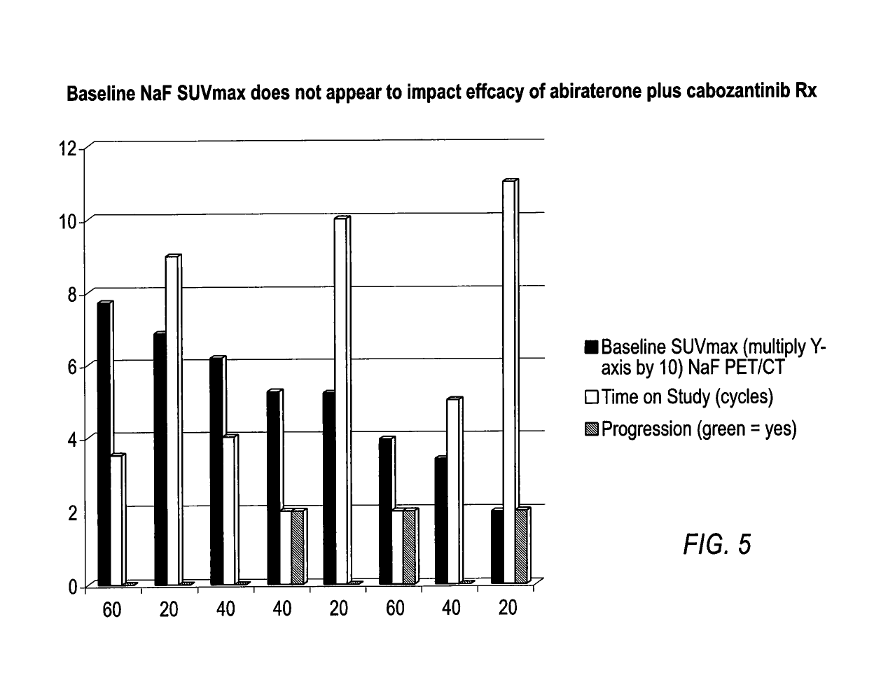

Baseline PET imaging results were measured for both ¹⁸F-FDG PET/CT and ¹⁸F—NaF PET/CT. FIG. 4 depicts the baseline PET imaging results for ¹⁸F-FDG PET/CT. The baseline elevated ¹⁸F-FDG PET/CT may identify the patients who do not benefit from abiraterone plus cabozantinib. FIG. 5 depicts the baseline PET imaging for ¹⁸F-NaF PET/CT. The baseline elevated ¹⁸F-NaF PET/CT does not appear to impact efficacy of abiraterone plus cabozantinib.

Twelve patients were measured to observe the differences between 20 mg and 40 mg of cabozantinib. Measures of SUVmax by PET/CT radiotracer (FDG and NaF) and PSA at baseline and after 8 weeks of treatment with cabozantinib and abiraterone, along with absolute and percentage changes were summarized using descriptive statistics (mean, standard deviation, median, and interquartile range). A two-sided paired t-test was used to assess the percent change in SUVmax levels for each radiotracer. There was little data to provide estimates of standard deviations of these measures in this patient population. Given 12 evaluable patients, there was 80% power to detect an effect of 0.89 times standard deviation, assuming a two-sided 5% significance level.

Based on a preliminary result in 5 patients, the NaF SUVmax mean percent change was a reduction of 36.5% (SD 25/9%). Thus, there is satisfactory power to detect a mean difference from baseline of 23%.

Patients were also dichotomized by FDG avid status at baseline, and the mean change in absolute SUVmax levels was compared between groups using a two-sided t-test. The effect size is defined as the difference between groups in mean change divided by a common SD. Assuming two-thirds of patients are FDG avid at baseline, there is 80% power to detect a difference of 1.9 times SD given a two-sided 5% significance level. For example, if the SD of change parallels the preliminary result for NaF SUVmax levels (SD=17), then there is satisfactory power to detect a difference in mean absolute change of 32 between FDG avid status groups.

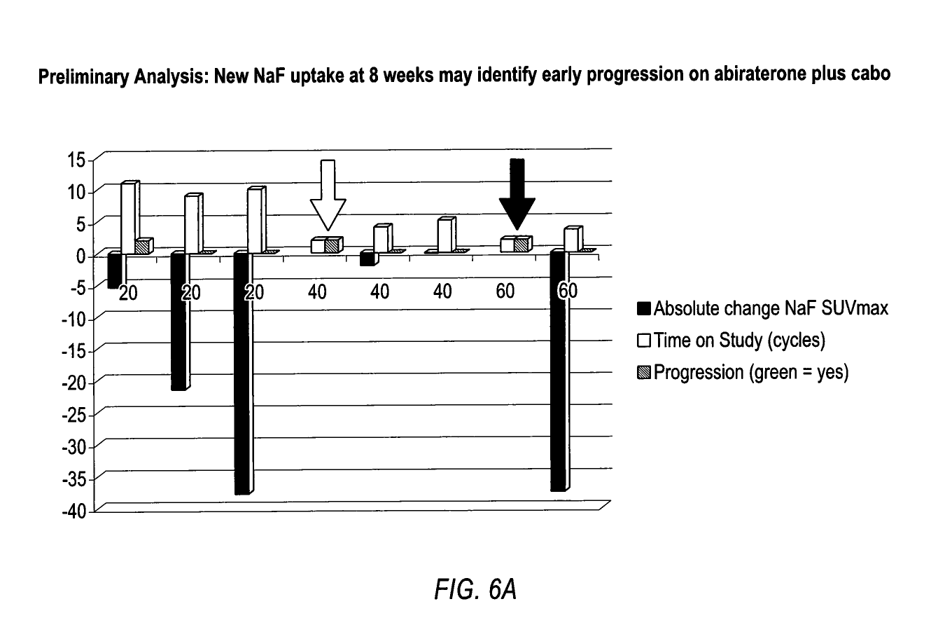

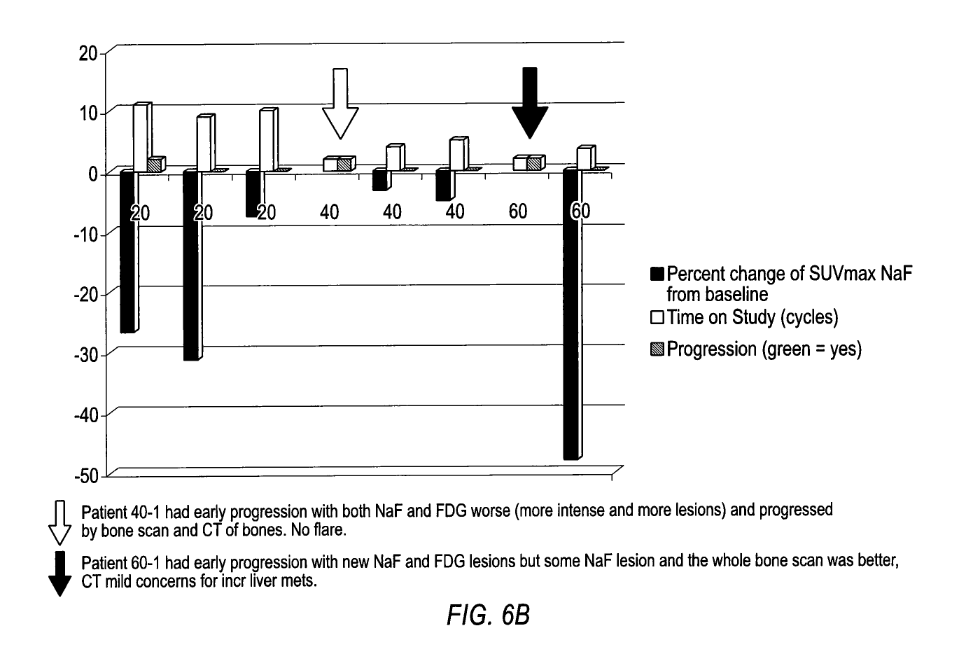

A preliminary analysis showed an increase of ¹⁸F-NaF PET/CT SUVmax at 8 weeks, which may identify early progression on abiraterone plus cabozantinib. FIG. 6 depicts these preliminary results. FIG. 6A shows the absolute change in ¹⁸F-NaF PET/CT SUVmax at 8 weeks. FIG. 6B shows the percent change of ¹⁸F-NaF PET/CT SUVmax from baseline at 8 weeks.

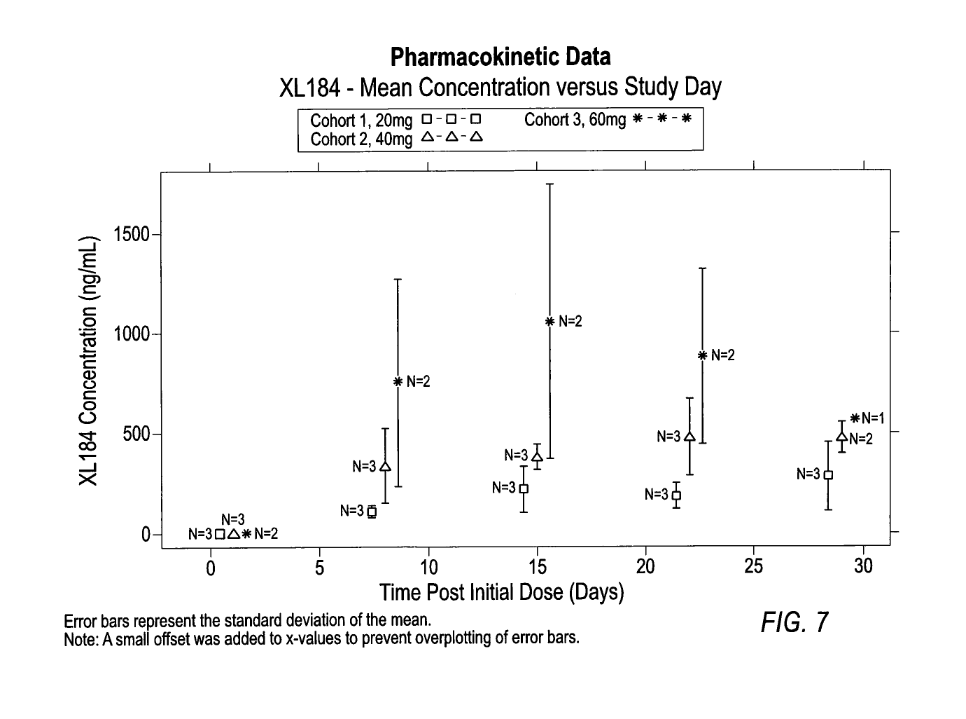

Pharmacokinetic data was also collected to determine the cabozantinib concentration for several days after the initial dose. FIG. 7 shows a chart of the mean concentration of cabozantinib versus the study day.

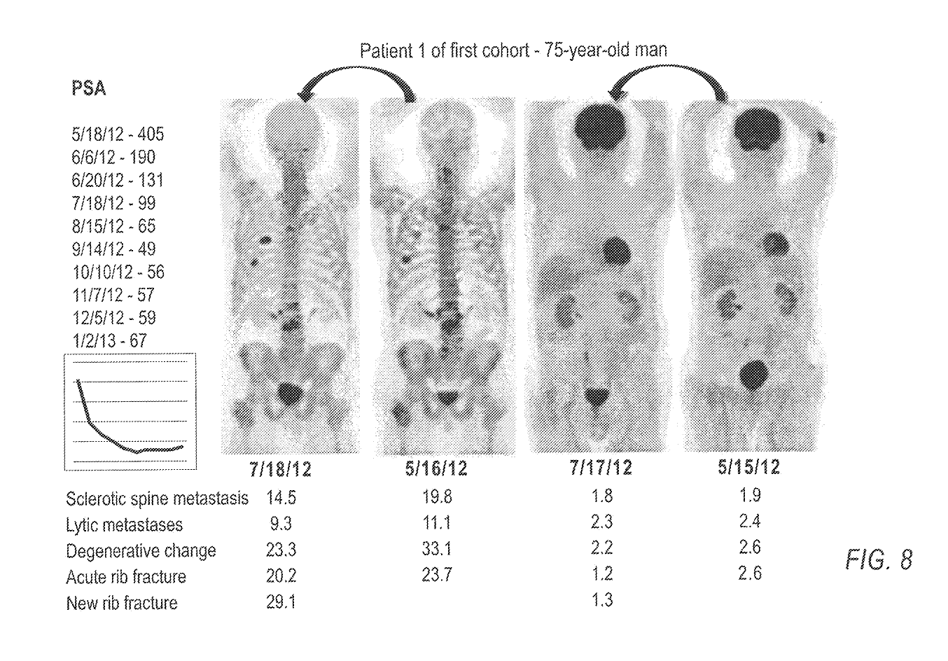

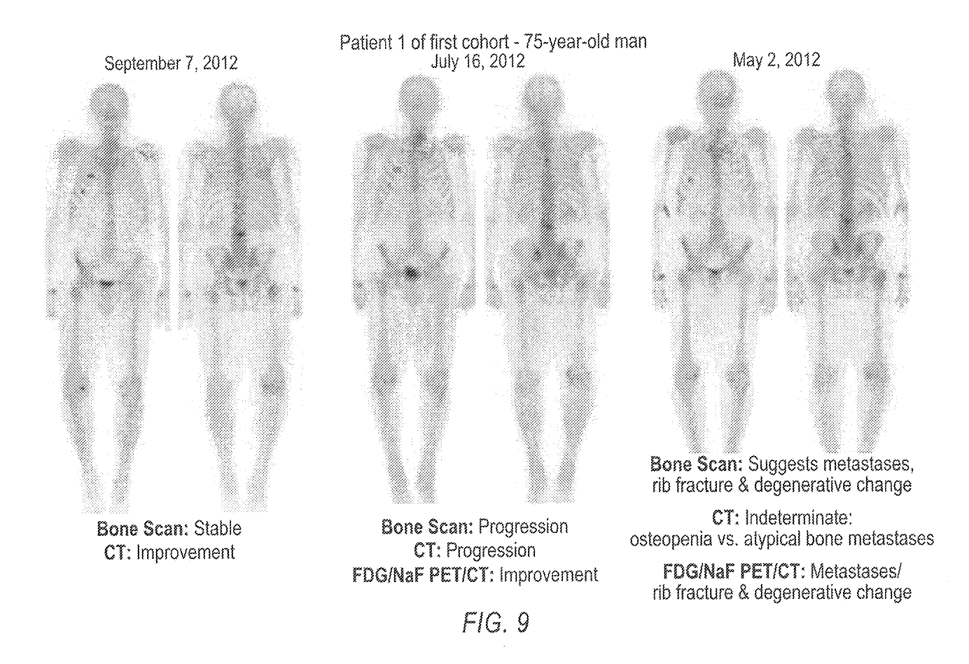

FIGS. 8 and 9 show the results for patient 1 of the first cohort, which received 20 mg cabozantinib. The patient was a 75-year old man with castrate resistant prostate cancer. FIG. 8 depicts the ¹⁸F-FDG PET/CT and ¹⁸F-NaF PET/CT scans at baseline and 8 weeks after the first dose. FIG. 9 shows full body bone scans at baseline, 8 months after the first dose, and 16 weeks after the first dose.

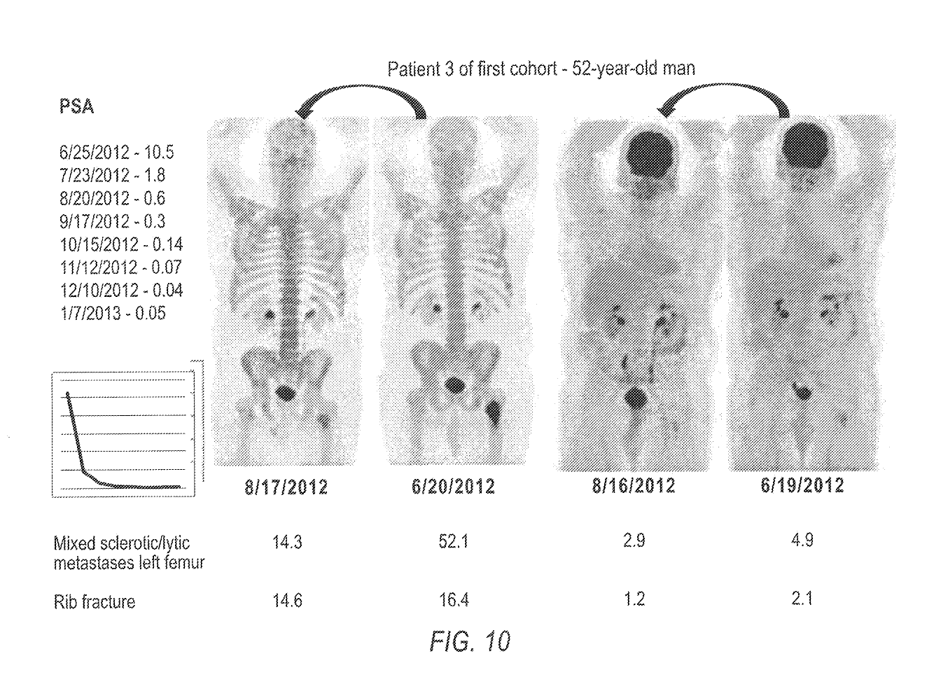

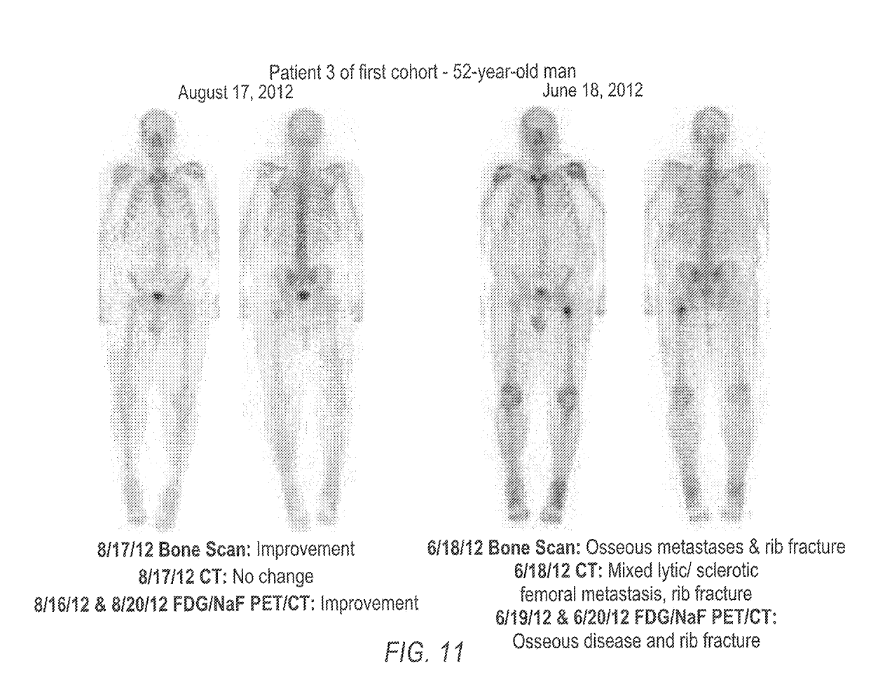

FIGS. 10 and 11 show the results for patient 3 of the first cohort, which received 20 mg cabozantinib. The patient was a 52-year old man with castrate resistant prostate cancer. FIG. 10 depicts the ¹⁸F-FDG PET/CT and ¹⁸F-NaF PET/CT scans at baseline and 8 weeks after the first dose. FIG. 11 shows full body bone scans at baseline and 8 months after the first dose.

The foregoing disclosure has been described in some detail by way of illustration and example for purposes of clarity and understanding. The invention has been described with reference to various specific and preferred embodiments and techniques. However, it should be understood that many variations and modifications can be made while remaining within the spirit and scope of the invention. It will be obvious to one of skill in the art that changes and modifications can be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

1. A method of treating cancer, comprising administering a patient in need of such treatment a compound of formula I: 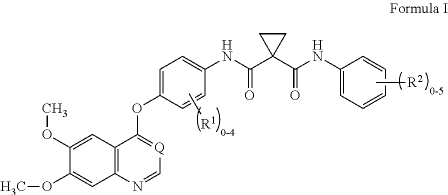
or a pharmaceutically acceptable salt thereof or a pharmaceutical composition comprising the compound of formula I and a pharmaceutically acceptable carrier, wherein:
R¹ is halo;
R² is halo; and
Q is CH or N;
in combination with a compound 2: 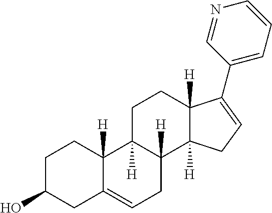
or a pharmaceutically acceptable salt thereof or a pharmaceutical composition comprising the compound of formula II and a pharmaceutically acceptable carrier.

2. The method of claim 1, wherein the compound of formula I is compound 1.

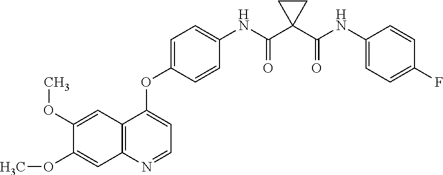 [SMILES: COc1cc2nccc(Oc3ccc(NC(=O)C4(C(=O)Nc5ccc(F)cc5)CC4)cc3)c2cc1OC]

3. The method of claims 1-2, wherein compound 1 is administered as the L-malate salt.

4. The method of claims 1-3 wherein compound 2 is administered as the acetate.

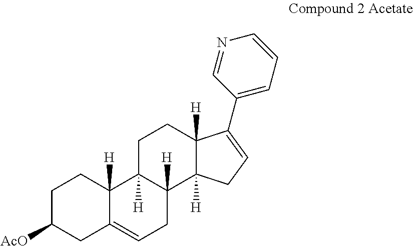

5. The method of claims 1-4, wherein the cancer is castration resistant prostate cancer.

6. The method of claims 1-5, wherein Compound 1 and Compound 2 are administered concurrently or sequentially.

7. The method of claims 1-6, wherein up to and including 1000 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

8. The method of claims 1-7, wherein up to and including 1000 mg of Compound 2 is administered to the patient once daily with fasting in combination with 60 mg, 40 mg, or 20 mg of compound 1 once daily with fasting.

9. The method of claims 1-8, wherein up to and including 750 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

10. The method of claims 1-9, wherein up to 750 mg of Compound 2 is administered to the patient once daily with fasting in combination with 60 mg, 40 mg, or 20 mg of compound 1 once daily with fasting.

11. The method of claims 1-10, wherein up to and including 500 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

12. The method of claims 1-11, wherein up to and including 500 mg of Compound 2 is administered to the patient once daily with fasting in combination with 60 mg, 40 mg, or 20 mg of compound 1 once daily with fasting.

13. The method of claims 1-12, wherein up to and including 250 mg of Compound 2 is administered to the patient once daily with fasting in combination with 100 mg, 95 mg, 90 mg, 85 mg, 80 mg, 75 mg, 70 mg, 65 mg, 60 mg, 55 mg, 50 mg, 45 mg, 40 mg, 35 mg, 30 mg, 25 mg, 20 mg, 15 mg, 10 mg, or 5 mg of compound 1 once daily with fasting.

14. The method of claims 1-13, wherein up to and including 250 mg of Compound 2 is administered to the patient once daily with fasting in combination with 60 mg, 40 mg, or 20 mg of compound 1 once daily with fasting.

15. The method of claims 1-14, further comprising prednisone or prednisolone.

16. The method of claims 1-15, further comprising 5 mg prednisone administered twice daily.

17. The method of claims 1-16, wherein a complete serological response is observed in patients being treated with the combination.

18. The method of claims 1-17, wherein a serological partial response is observed in patients being treated with the combination.

19. The method of claims 1-18, wherein stable disease is observed in patients being treated with the combination.
